(19) United States
(12) Patent Application Publication
(10) Pub. No.: US 2026/0242760 A1
(43) Pub. Date: Aug. 20, 2026

(54) COMPOSITIONS AND METHODS OF ANCESTRAL CRISPR-CAS ENZYMES

(71) Applicant: Board of Trustees, Southern Illinois University, Carbondale, IL (US)

(72) Inventors: Keith T. Gagnon, Pfafftown, NC (US); Christopher L. Barkau, Carbondale, IL (US); Adam A. White, Winston-Salem, NC (US)

(21) Appl. No.: 19/366,360

(22) Filed: Oct. 22, 2025

Related U.S. Application Data
(60) Provisional application No. 63/710,408, filed on Oct. 22, 2024.

(51) Int. Cl. C12N 9/22; C12N 15/11; C12N 15/86; C12Q 1/34; C12Q 1/6897; G16B 10/00; G16B 15/20; G16B 30/10

(52) U.S. Cl. CPC C12N 9/226; C12N 15/111; C12N 15/86; C12Q 1/34; C12Q 1/6897; G16B 10/00; G16B 15/20; G16B 30/10; C12N 2310/20; C12N 2740/15043; G01N 2333/922

(57) ABSTRACT

A method is provided for identifying and creating novel ancestral AncCas proteins comprising performing ancestral sequence reconstruction, performing a computational analysis of predicted sequences, conducting in vitro biochemical assays; and conducting cell-based assays. A composition is also provided comprising an AncCas protein, wherein the AncCas protein comprises an amino acid sequence having a sequence similarity of at least 95% with respect to at least one sequence selected from the group consisting of SEQ. ID. NOs. 1-10 and functional fragments thereof. The composition includes a guide RNA where the guide RNA comprises a nucleotide sequence having a sequence similarity of at least about 95% with respect to at least one sequence selected from the group consisting of SEQ. ID. NOS. 11-22 and functional fragments thereof. The composition includes a SNuB wherein the SNuB comprises a nucleotide sequence having a sequence similarity of at least 95% with respect to SEQ. ID. NO. 23.

### CROSS REFERENCE TO RELATED APPLICATION

This application claims priority to U.S. Provisional Application No. 63/710,408, filed on Oct. 22, 2024, which hereby is incorporated herein by reference in its entirety.

### SEQUENCE LISTING

This application contains a sequence listing filed in electronic form as an .xml file entitled “500977-180.xml” created on Apr. 24, 2026, and has a size of 68.0 kb. The content of the sequence listing is incorporated herein in its entirety.

### FIELD OF THE INVENTION

The present disclosure relates to compositions and methods of novel ancestral CRISPR-Cas Enzymes. More particularly, the present disclosure relates to a method for developing novel ancestral Cas proteins and compositions for genetic modulation including ancestral Cas proteins.

### BACKGROUND OF THE INVENTION

Clustered regularly interspaced short palindromic repeats (CRISPR) and associated Cas (CRISPR-associated) proteins constitute the CRISPR-Cas system. This system originated in bacteria as a defense mechanism against bacteriophages. Cas enzymes are DNA endonucleases, which utilize a RNA dual guide system to specifically target and cleave DNA in a sequence-dependent manner. The dual guide system is comprised of a CRISPR RNA (crRNA) which is bound to a trans-activated crRNA (tracrRNA).

Researchers have attempted to adapt the CRISPR-Cas systems (“CRISPR”) to aid in treatment of genetic diseases, an approach now at the forefront of potential gene therapy strategies. CRISPR has potential applications in treating genetic diseases, diagnostics and improving the properties of agricultural cultivars.

CRISPR-Cas9 from Streptococcus pyogenes (SpCas9) was among the first CRISPR-Cas system discovered, characterized, and developed for gene editing. It has since become the model, prototypical platform for novel biotechnology and therapeutic development. These include a wide variety of derivatives with altered protospacer-adjacent motif (PAM) sequence preferences, truncated sizes, and protein fusions. Although a variety of novel CRISPR-Cas9 systems have also been identified and characterized from other organisms, the utility and success of SpCas9 has been unrivaled.

Despite its success, therapeutic development of SpCas9 has struggled with off-target safety profiles, flexibility in PAM preference, delivery modalities, and immunogenic potential in patients. This has not been easily remedied by identification and characterization of other existing CRISPR-Cas enzymes, such as through metagenomic and phylogenetic approaches.

Thus, there is a need to identify novel CRISPR-Cas enzymes.

### SUMMARY

In one embodiment, a composition comprising an AncCas protein, wherein the AncCas protein comprises an amino acid sequence having a sequence similarity of at least about 95% with respect to at least one sequence selected from the group consisting of SEQ. ID. NOs. 1-10 and functional fragments thereof.

In one embodiment, a composition further comprising an AncCas protein, wherein the AncCas protein comprises an amino acid sequence having a sequence similarity of at least about 95% with respect to at least one sequence selected from the group consisting of SEQ. ID. NOs. 1-10.

In one embodiment, the composition further comprising the AncCas protein wherein the protein has a predicted SP of about 0.2 to about 1.0. In one embodiment about 0.4 to about 0.8. In one embodiment about 0.5 to about 0.7.

In one embodiment, the composition further comprising wherein the AncCas protein has a sequence similarity of at least about 70% with respect to SpCas9.

In one embodiment, a composition further comprising a guide RNA (e.g., sgRNA).

In one embodiment, a composition further comprising wherein the guide RNA (e.g., sgRNA) comprises a chemically modified guide RNA (e.g., a chemically modified sgRNA).

In one embodiment, a composition further comprising wherein the guide RNA (e.g., sgRNA) comprises a nucleotide sequence having a sequence similarity of at least about 95% with respect to at least one sequence selected from the group consisting of SEQ. ID. NOS. 16-22 and functional fragments thereof. In one embodiment, having a sequence similarity of at least about 90%. In one embodiment, having a sequence similarity of at least about 97%. In one embodiment, having a sequence similarity of at least about 99%.

In one embodiment, the composition further comprising wherein the guide RNA (e.g., sgRNA) comprises a nucleotide sequence having a sequence similarity of at least about 95% with respect to at least one sequence selected from the group consisting of SEQ. ID. NOS. 16-22. In one embodiment, having a sequence similarity of at least about 90%. In one embodiment, having a sequence similarity of at least about 97%. In one embodiment, having a sequence similarity of at least about 99%.

In one embodiment, the composition further comprising wherein the composition further comprises an Anti-PAM small nucleic acid-based inhibitor (SNuB) or the composition is compatible with a small nucleic acid-based inhibitor (SNuB).

In one embodiment, the composition further comprising wherein the SNuB comprises a nucleotide sequence having a sequence similarity of at least about 95% with respect to at least one sequence selected from the group consisting of SEQ. ID. NO. 23 and functional fragments thereof. In one embodiment, selected from the group consisting of SEQ. ID. NO. 23. In one embodiment, having a sequence similarity of at least about 90%. In one embodiment, having a sequence similarity of at least about 97%. In one embodiment, having a sequence similarity of at least about 99%.

In one embodiment, the composition further comprising wherein the SNuB comprises a nucleotide sequence having a sequence similarity of at least about 95% with respect to SEQ. ID. NO. 23.

In one embodiment, the composition further comprising wherein the composition selectively alters expression of at least one gene product or the composition selectively alters activity of at least one AncCas protein-guide RNA system.

In one embodiment, a method for identifying and creating novel ancestral CAS (AncCas) proteins comprising: performing ancestral sequence reconstruction; performing a computational analysis of predicted sequences; conducting an in vitro biochemical assay; and conducting an in vivo cell-based assay.

In one embodiment, the method for identifying and creating novel ancestral CAS (AncCas) proteins further comprising wherein said performing ancestral sequence reconstruction comprises: accumulating and aligning extant Cas sequences; generating a phylogenetic tree; and using an ASR algorithm software.

In one embodiment, the method for identifying and creating novel ancestral CAS (AncCas) proteins further comprising wherein said generating a phylogenetic tree comprises a phylogenetic analysis including protein sequences comprising at least 60 Cas protein sequences (e.g., Streptococcus Cas protein sequences). In one embodiment, between 5 and 5,000,000 Cas protein sequences (e.g., Streptococcus Cas protein sequences).

In one embodiment, the method for identifying and creating novel ancestral CAS (AncCas) proteins further comprising wherein said performing a computational analysis of predicted sequences comprises at least one of: predicting protein structure; predicting protein solubility when expressed in bacterial (e.g., E. coli) or mammalian host cells; predicting protein stability and charge; predicting the flexibility of residues in the resulting protein; and combinations thereof.

In one embodiment, the method for identifying and creating novel ancestral CAS (AncCas) proteins further comprising wherein said conducting an in vitro biochemical assay comprises at least one of: cloning and purification of a candidate AncCas sequence into a protein expression vector; conducting a PAM finding assay (e.g., using next generation sequencing (NGS)); assessing chemically modified guide cleavage activity; assessing inhibition from studied small nucleic acid-based inhibitors (SNuBs); and combinations thereof.

In one embodiment, the method for identifying and creating novel ancestral CAS (AncCas) proteins further comprising wherein said conducting an in vivo cell-based assay comprises at least one of: performing transfection of assembled RNP complexes; performing PCR subcloning of the selected AncCas gene into a lentiviral vector; performing transfection of the AncCas encoded plasmid for fluorescence gene knockdown; and combinations thereof.

In one embodiment, the method for identifying and creating novel ancestral CAS (AncCas) proteins further comprising wherein at least one of the resulting AncCas proteins comprises an amino acid sequence having a sequence similarity of at least about 95% with respect to at least one sequence selected from the group consisting of SEQ. ID. NOs. 1-10 and functional fragments thereof. In one embodiment, selected from the group consisting of SEQ. ID. NOs. 1-10.

In one embodiment, the method for identifying and creating novel ancestral CAS (AncCas) proteins further comprising wherein the AncCas protein has a predicted SP of about 0.2 to about 1.0. In one embodiment about 0.4 to about 0.8. In one embodiment about 0.5 to about 0.7.

In one embodiment, the method for identifying and creating novel ancestral CAS (AncCas) proteins further comprising wherein the AncCas protein has a sequence similarity of at least about 70% with respect to SpCas9.

In one embodiment, a method of selectively modulating gene activity comprising administering a composition comprising a AncCas protein and a guide RNA (e.g., sgRNA).

In one embodiment, a method of selectively modulating gene activity further comprising wherein the AncCas protein comprises an amino acid sequence having a sequence similarity of at least about 95% with respect to at least one sequence selected from the group consisting of SEQ. ID. NOs. 1-10 and functional fragments thereof. In one embodiment, selected from the group consisting of SEQ. ID. NOs. 1-10.

In one embodiment, a method of selectively modulating gene activity further comprising wherein the guide RNA (e.g., sgRNA) comprises a nucleotide sequence having a sequence similarity of at least about 95% with respect to at least one sequence selected from the group consisting of SEQ. ID. NOS. 11-22.

In one embodiment, a method of selectively modulating gene activity further comprising wherein the AncCas and guide RNA (e.g., sgRNA) are administered with a SNuB.

### DESCRIPTION OF THE DRAWINGS

FIGS. 1A and 1B are an illustration of an exemplary method for characterizing novel proteins produced through ancestral state reconstruction.

FIG. 2 is an illustration of an exemplary theory of the method of ASR and the generation of ancestral protein sequences.

FIGS. 3A-3D include graphs and illustrations of exemplary analyses of predicted sequences including analysis of ionic strength over pH (heatmaps), solubility predictions, and Alphafold structural predictions.

FIGS. 4A and 4B are an illustration of an exemplary phylogenetic analysis of ancestral Cas9 protein sequences and extant Cas9 protein sequences as well as an exemplary structural overlay of predicted protein structures of exemplary ancestral Cas9 protein sequences and extant Cas9 protein sequences.

FIG. 5 is a graph showing in vitro cleavage of exemplary ancestral Cas9 (AncCas) proteins and extant Cas9 (ExtCas) proteins.

FIG. 6A is an illustration demonstrating structure deviation of an exemplary AncCas protein compared to an ExtCas protein and FIG. 6B illustrates the sequence similarity between an exemplary AncCas protein sequence and an ExtCas protein sequence.

FIG. 7 is a graph showing anti-Cas9 antibody detection of exemplary AncCas proteins compared to an ExtCas protein.

FIGS. 8A-8C are an illustration of an exemplary protospacer adjacent motif (PAM) finding assay and exemplary results indicating conservation of PAM site in exemplary ancestral Cas9 enzymes with minimal relaxation of sequence preference.

FIG. 9A is an illustration of an exemplary human cell model system showing a ribonucleoprotein (RNP) assembly and knockout of the GFP gene to turn off fluorescence in HEK293T cells, FIG. 9B is a graph of exemplary flow cytometry results showing that exemplary AncCas proteins are capable of RNP complex editing of the GFP gene in HEK293T cells, FIG. 9C includes exemplary bright field and green fluorescence images showing that exemplary AncCas proteins are capable of knocking out GFP in HEK293T cells, and FIG. 9D is a graph of exemplary flow cytometry results showing that exemplary AncCas proteins are capable of lentiviral transfection cell editing.

FIG. 10A includes an illustration of an exemplary alignment of AncCas-114 related ExtCas guide RNAsgRNA sequences identified by reference genomic sequence location and FIG. 10B is an exemplary consensus sgAncCas-114 RNA as inferred from extant sgRNAs.

FIGS. 11A and 11B illustrate the secondary structures of an exemplary proposed sgRNA sequence of optimized Streptococcus pyogenes Cas9 and an exemplary designed sgAncCas-114.

FIGS. 12A and 12B include exemplary graphs showing in vitro cleavage assay results using canonical and inferred sgRNAs, demonstrating the efficiency to cleave dsDNA between ExtCas-53 and exemplary AncCas-114.

FIG. 13 includes an illustration showing an exemplary fused single guide RNA (sgRNA) system for use by Cas9 or exemplary AncCas proteins.

FIG. 14 includes an illustration showing an exemplary dual guide RNA (dgRNA) system for use by Cas9 or exemplary AncCas proteins.

FIG. 15 includes an illustration of various exemplary chemically modified guide RNAs for use with exemplary AncCas proteins.

FIG. 16 includes a graph showing in vitro cleavage assay results for ExtCas-53 (SpCas9) and exemplary AncCas-114 Cas proteins using various chemically modified guides.

FIG. 17 illustrates an exemplary sequence and structure of a small nucleic acid-based inhibitor (SNuB).

FIG. 18 is a graph showing in vitro cleavage inhibition of spCas9 and exemplary AncCas-114 Cas protein by the SNuB inhibitors of FIG. 17.

FIGS. 19A-19I include illustrations of predicted protein folding structures of various exemplary AncCas proteins. FIG. 19A illustrates the alphafold predicted structure for AncCas-81. FIG. 19B illustrates the alphafold predicted structure for AncCas-84. FIG. 19C illustrates the alphafold predicted structure for AncCas-87. FIG. 19D illustrates the alphafold predicted structure for AncCas-112. FIG. 19E illustrates the alphafold predicted structure for AncCas-114. FIG. 19F illustrates the alphafold predicted structure for AncCas-120. FIG. 19G illustrates the alphafold predicted structure for AncCas-121. FIG. 19H illustrates the alphafold predicted structure for AncCas-123. FIG. 19I illustrates the alphafold predicted structure for AncCas-124.

### DETAILED DESCRIPTION

Before any examples of the disclosure are explained in detail, it is to be understood that the disclosure is not limited in its application to the details of construction and the arrangement of components set forth in the following description or illustrated in the following drawings. The disclosure is capable of other examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The following discussion is presented to enable a person skilled in the art to make and use examples of the disclosure. Various modifications to the illustrated examples will be readily apparent to those skilled in the art, and the generic principles herein is applied to other examples and applications without departing from examples of the disclosure. Thus, examples of the disclosure are not intended to be limited to examples shown but are to be accorded the widest scope consistent with the principles and features disclosed herein. The following detailed description is to be read with reference to the figures, in which like elements in different figures have like reference numerals. The figures depict selected examples and are not intended to limit the scope of examples of the disclosure. Skilled artisans will recognize the examples provided herein have many useful alternatives and fall within the scope of examples of the disclosure.

CRISPR-associated (Cas) proteins and their CRISPR RNA (crRNA) guides are used for programmable gene and RNA editing, and ultimately human gene therapy. The traditional CRISPR-Cas system most used consists of a singular effector protein, Cas9 from Streptococcus pyogenes (SpCas9), and a fusion of its two natural guide RNAs: the CRISPR RNA (crRNA) and trans-activating crRNA (tracrRNA), into a single-guide RNA (sgRNA). These ribonucleoprotein (RNP) complexes can recognize and bind double-stranded DNA (dsDNA) and cause double-strand breaks (DSBs). In mammalian cells, DSBs can either cause functional gene knockout or can allow insertion of donor DNA when they are repaired. Cas9 has been modified and leveraged in a wide variety of ways to bind DNA or RNA, edit bases, or add epigenetic modifications. CRISPR-Cas systems is used for therapeutic applications, including ex vivo and in vivo gene editing. Another enzyme, Cas12a, has also been investigated for its potential in therapeutics. Cas 12a holds therapeutic potential as a smaller, uniquely folded protein with a different PAM preference and a naturally occurring single crRNA. Other CRISPR-Cas systems, such as CRISPR-Cas13, have been characterized to sequence-specifically bind and cut RNA. For therapeutics and research, these proteins have the potential to orthogonally manipulate RNA metabolism in cells, but some challenges in their development involve unintended collateral cleavage of non-target RNAs and potential immunogenic issues linked to the need for long-term expression.

Currently, there are many limitations of the current CRISPR-Cas systems for therapeutic gene editing. The utility of available CRISPR-Cas enzymes is limited by the characteristics of the few that have been studied or found to function in human cells. Some limitations of the current systems also include sequence specificity, PAM preference, and immunogenicity. Sequence specificity is directly linked to off-target editing, which can cause unwanted side-effects, possibly including cancer. While efforts to engineer more specific SpCas9, for example, have uncovered important insights into catalytic mechanisms, a deeper understanding of the relationship between Cas mechanisms and sequence specificity will be required to unlock better engineering. Regarding PAM preferences, Cas9 proteins tend to have guanine-rich PAM preferences and Cas 12a proteins prefer adenine/thymine-rich PAMs, which can limit the targets within the human genome.

Some currently engineered derivatives of Cas9 include xCas9, SpRY, and SpG, however these derivatives can exhibit reduced PAM requirements, and can also compromise activity or specificity. Additional natural, extant (existing) Cas9 enzymes have also been characterized with more diverse PAM preferences, but these extant Cas9 enzymes often can lack high editing efficiency or can possess overly complex PAM requirements.

Existing, or extant, Cas9 proteins have been found to elicit immune responses when introduced into animals. This is especially true for the leading SpCas9 system, where a large fraction of the human population, and by extension potential patients, possess antibodies against SpCas9. Thus, Cas proteins that cannot elicit an immune response upon initial introduction into patients would significantly improve therapeutic safety and broad utility. Ancestral Cas proteins fill this need.

Despite the diversity of known Cas homologs, relatively few have been identified as suitable for gene editing. In one embodiment, a large screen of Cas12a enzymes, found that only two out of sixteen of the Cas12a enzymes could successfully edit human cells. Thus, because of the low success rate of screening for gene editors, there is a need for better predictive methods with more promising starting points.

Currently, the conventional method for identifying new CRISPR-Cas enzymes is through searching sequences that already exist in genome databases or by incorporating alternative sequence modifications followed by producing and testing the enzyme constructs for functional activity.

Development of a more promising method for identifying new Cas systems is challenging since very few amino acid changes are possible without negatively impacting activity. Thus, the method design requires deep understanding of sequence-structure-function-evolution and how to effectively explore that space, therefore it is challenging to engineer diverse, functional enzymes.

According to the teachings therein, a method for identifying and novel ancestral CRISPR-Cas enzymes is provided.

Additionally, a method for ancestral sequence reconstruction (ASR) of Cas proteins and CRISPR RNA is provided.

Furthermore, compositions including ancestral Cas proteins and methods of use are provided herein.

In one embodiment, the provided ASR method can discover unique CRISPR-Cas enzymes that are functional, possessing either new or improved properties or the ability to serve as convenient replacements or alternatives to current CRISPR-Cas systems in use.

In one embodiment, a method for identifying new Cas proteins includes use of model-based methods for example maximum likelihood (ML) and Bayesian analysis to determine molecular phylogenetics.

In one embodiment, the method can determine the relationship between molecular evolution to biological function.

In one embodiment, ML and Bayesian analysis can also be extended to apply ASR and can infer the sequence of internal nodes in phylogenetic trees, representing ancestral genes. In one embodiment, very little knowledge of enzyme properties is required for ASR. The ASR method can infer the sequences of proteins that once existed and served a function. Thus, the ASR method serves as a relatively safe guide for navigating alternative sequence space. The disclosed method using ASR avoids the need for use of massive mutagenesis or in vitro evolution screens, allowing millions of years of inferred evolution do the heavy lifting. In an example, the fidelity of inferences made by the method are robust to phylogenetic uncertainty.

Demonstrated herein is ASR technology developed for the successful application to CRISPR-Cas systems to produce functional enzymes.

According to the teachings therein, a novel CRISPR-Cas enzyme discovery pipeline is provided wherein the pipeline includes a method which combines new and traditional approaches for example, phylogenetics, ASR, computational predictions, high throughput assays, biochemistry, and cellular and molecular biology. In one embodiment the method can analyze evolutionary time and sequence space at a breadth not possible by other current methods for identifying new Cas proteins.

In one embodiment, the method involves creating a phylogenetic tree of known CRISPR-Cas sequences then performing ancestral state reconstruction (ASR) analyses to predict the Cas protein and CRISPR RNA sequences of putative ancestors of the extant (existing) known CRISPR-Cas systems. In one embodiment, the ancestor reconstruction is only predictive, not definitive, therefore the enzymes identified may not previously have existed in nature. Once putative ancestral sequences are identified, they are bioinformatically screened for predicted structural folding, stability, and any other suitable properties. Next, proteins are produced in the laboratory and tested for biochemical properties, including guide RNA preference, catalytic activity, protospacer adjacent motif (PAM) requirements, and ability to perform gene or RNA editing in human cells.

In one embodiment, the method herein is used to identify and produce novel CRISPR-Cas enzymes that are functional, possessing either new or improved properties or the ability to serve as convenient replacements or alternatives to current CRISPR-Cas systems in use.

The methods as described In one embodiment have demonstrated utility by identifying and producing a novel ancestral Cas9 protein named AncCas-114. AncCas-114 can function at similar specificity and activity as SpCas9. In one embodiment, AncCas-114 has a sequence similarity 72% with respect to SpCas9 and is not recognized by anti-SpCas9 antibody. AncCas-114 also can use various chemically modified crRNAs and nucleic acid-based inhibitors more efficiently than SpCas9, which is relevant for therapeutic development of CRISPR-Cas9. In one embodiment, the method is amenable to identification and production of other classes of CRISPR-Cas enzymes, such as Cas12a and Cas13. In one embodiment, the method could be used in any CRISPR-Cas system including Cas9, Cas12a, Cas13 enzymes, Fanzors, and other related or unrelated RNA-guided enzymes.

In one embodiment, the predicted enzymes deviate from existing enzymes by at least about 20% in sequence similarity. In additional examples, the predicted enzymes deviate from the existing enzymes by at least about 5%, at least about 10%, at least about 15%, at least about 20%, at least about 30%, or at least about 40%.

In one embodiment, a composition is provided including any AncCas protein. In another example, a AncCas-114 protein is provided. The AncCas9-114 enzyme can exhibit the same NGG PAM preference, high editing efficiency, and guide RNA requirements as SpCas9. However, AncCas9-114 can possess less than 80% sequence similarity to SpCas9. The sequence divergence from SpCas9 and the lack of existence in modern organisms show that AncCas9-114, and other AncCas proteins with even less sequence similarity, could reduce or eliminate immunogenicity. AncCas9-114 is designed to use heavily modified guide RNAs and modified small nucleic acid-based inhibitors (SNuBs), both of which can advance therapeutic translation of CRISPR-Cas9 systems.

The disclosed method for identifying and producing novel CRISPR-Cas enzymes can require using a minimum number of known existing and relatively closely related CRISPR-Cas enzyme sequences to be used to construct a robust phylogenetic tree for CRISPR-Cas enzyme prediction. The resulting predicted CRISPR-Cas enzyme systems are created and tested. In one embodiment, the resulting successful systems provide improved performance over, or interchangeably with, existing Cas9, Cas12, Cas13, Fanzor, TnpB, IscB, IS110 or other related RNA-guided and CRISPR-Cas systems currently in use or being developed for research, therapeutics, diagnostics, and other biotechnology applications.

In one embodiment, the method as described herein includes a pipeline which is amenable to a broad array of RNA guided enzymes and their derivatives, allowing for discovery and creation of novel CRISPR-Cas systems that improve clinical utility and democratize access to development.

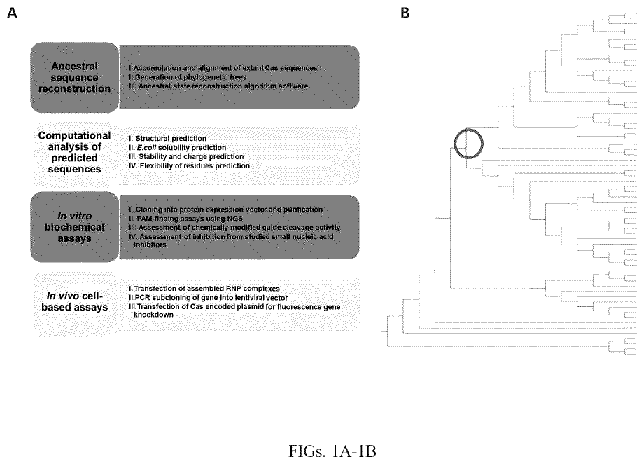

Turning to FIG. 1A, a method of identifying and creating Cas constructs is provided. The method identifies and produces novel ancestral Cas (AncCas) proteins. In one embodiment, the method includes a single step of performing ancestral sequence reconstruction (ASR).

In one embodiment, the method includes the step of performing ASR and a step of performing computational analysis of predicted sequences.

In one embodiment, the method includes the step of performing ASR, the step of performing computational analysis of predicted sequences, a step of conducting in vitro biochemical assays, and a step of conducting in vivo cell-based assays, and combinations thereof.

In one embodiment, the step of performing ASR includes accumulating and aligning extant Cas sequences, generating a phylogenetic tree, and using an ASR algorithm software.

In one embodiment, the step of performing ASR includes using ProtASR2, which can perform inference and ASR with realistic thermodynamic properties.

In one embodiment, the ASR algorithm software comprises ProtASR2 or any other software known to those skilled in the art.

In one embodiment, the step of performing ASR includes using ASR in a phylogenetics package or a dedicated ASR tool.

In one embodiment, the step of performing computational analysis of predicted sequences includes predicting protein structure, predicting protein solubility when expressed in bacterial (e.g., E. coli) or mammalian host cells predicting protein stability and charge, and predicting the flexibility of residues in the resulting protein.

In one embodiment, predicting protein structure includes predicting the three-dimensional structures of AncCas proteins using AlphaFold2 or any other software known to those skilled in the art.

In one embodiment, predicting protein solubility when expressed in bacterial (e.g., E. coli) or mammalian host cells is performed using SoluProt, or any other suitable software, to predict AncCas solubility when expressed in E. coli.

In one embodiment, predicting the stability and charge, in the resulting protein is accomplished using Protein-Sol, or any other software known to those skilled in the art.

In one embodiment, MEDUSA9, or any other software known to those skilled in the art, is used to predict the flexibility of residues across the protein structures.

In one embodiment, after in-silico evaluation, candidate AncCas sequences is selected for in vitro characterization via biochemical assays. Selection criteria for candidate AncCas sequences includes at least one of predicted structure and solubility, proximity to extant proteins selected for characterization, and distance from the phylogenetic tree root.

In one embodiment, the step of conducting in vitro biochemical assays includes at least one of cloning the candidate AncCas sequence into a protein expression vector and purification, conducting PAM finding assays (e.g., using next generation sequencing (NGS)), assessing chemically modified guide cleavage activity, and assessing inhibition from studied small nucleic acid-based inhibitors (SNuBs).

In one embodiment, cloning the selected AncCas sequence into a protein expression vector and purifying the AncCas protein includes synthesizing genes and purifying the resulting protein using any suitable method in the art and/or commercial vendors.

In one embodiment, selected AncCas sequences is cloned into a pET28a plasmid, expression vector, or any other suitable plasmid in the art for expression.

In one embodiment, the purification of the plasmid, expression vector, or any other suitable plasmid in the art for expression is achieved using conventional purification and detection tags such as 6×His, HA, and 3×NLS or any other suitable methods.

In one embodiment, conducting PAM finding assays (e.g., using NGS) includes assembling AncCas proteins of interest with candidate guide RNAs and combining the Cas-gRNA with a target DNA duplex comprising the cognate target sequence of the guide RNAs adjacent to a randomized region.

In one embodiment, the guide RNA scaffolds for the AncCas proteins are determined by multiplex activity and specificity assays using RNA guides from extant systems that represent the sequence or structural diversity of guides from the group.

Furthermore, In one embodiment, conducting PAM finding assays (e.g., using NGS) includes using sequencing results to identify PAM preferences for each AncCas protein.

In one embodiment, assessing the in vitro cleavage activity of exemplary AncCas proteins with various chemically modified guides includes using an assay combining the AncCas, the guide RNA, and a linearized EGFP plasmid followed by resolution of products on an agarose gel and analysis of cleavage fractions.

In one embodiment, the in vitro cleavage testing includes performing multiplexed gRNA testing.

In one embodiment the step of conducting in vitro biochemical assays includes at least performing time courses, titrations, and trans and collateral cleavage activity assays. Promiscuous non-target cleavage activity is characterized using quenched fluorescence proximity assays or any other suitable assay as known in the art.

In general, non-target cleavage activity is characterized using methods known to those skilled in the art.

In one embodiment, the step of conducting in vitro biochemical assays further includes performing enzyme-linked immunosorbent assay (ELISA) testing for antibody binding to AncCas proteins. In one embodiment, performing ELISA testing for antibody binding to AncCas proteins includes measuring antibody binding of SpCas9 to exemplary AncCas proteins.

In one embodiment, assessing inhibition from studied small nucleic acid-based inhibitors (SNuBs) includes performing in vitro cleavage reactions containing the exemplary AncCas as well as the necessary guide RNA for cleavage as well as an exemplary SNuB.

In one embodiment of AncCas systems described herein, addition of at least one SNuB can result in substantial inhibition of cleavage activity.

In one embodiment, the step of conducting in vivo cell-based assays includes at least performing transfection of assembled RNP complexes, performing PCR subcloning of the selected AncCas gene into a lentiviral vector, and performing transfection of the AncCas encoded plasmid for fluorescence gene knockdown.

In one embodiment, performing transfection of assembled RNP complexes is performed using any suitable methods. In general, transfection of assembled RNP complexes is performed using any suitable assay as known in the art.

In one embodiment, performing transfection includes assembling RNP complexes by combining purified AncCas proteins with at least a guide RNA targeting a model EGFP gene which is expressed constitutively in HE293T cells or any other suitable cells. Next, the RNP is electroporated into the HE293T cells using a neon transfection instrument. Further, editing of the EGFP gene is quantified by the loss of EGFP fluorescence using flow cytometry or any other suitable method for measuring fluorescence. In general, editing of the EGFP gene is quantified using any suitable assay as known in the art.

In one embodiment, performing PCR subcloning of the selected AncCas gene into a lentiviral vector is performed using any suitable methods known in the art.

In one embodiment, performing PCR subcloning includes subcloning into a custom pLJM1 mammalian expression vector.

In one embodiment, cloning success and sequence integrity is verified by nanopore sequencing.

In one embodiment, a mammalian expression vector and a GFP targeting guide RNA (e.g., sgRNA) are transfected into cells and resulting cells are analyzed by microscopy and flow cytometry to determine knockout efficiency

In one embodiment, performing transfection of the AncCas encoded plasmid for fluorescence gene knockdown is performed using any suitable methods known in the art.

In one embodiment, AncCas encoded plasmids are subcloned into lentiviral vectors for transfection and expression in mammalian cell models.

In one embodiment, the step of conducting in vivo cell-based assays includes at least targeting of endogenous genes (for example VEGF or EMX1).

In one embodiment, the method of identifying and creating novel ancestral CAS (AncCas) proteins includes a step of analyzing substrate specificity and off-target editing potential.

In one embodiment, the step of analyzing substrate specificity and off-target editing potential includes analyzing AncCas enzymes that show suitable or high editing activity in human cells using any suitable in-silico analysis.

In one embodiment, the in-silico analysis includes prediction of potential off-target sites using Cas-OFFinder or any other suitable software known in the art.

In one embodiment, the in-silico analysis to determine substrate specificity and off-target editing potential includes using CIRCLE-seq, or CASowary followed by RT-qPCR.

Referring now to FIG. 1B, the method for identifying and creating novel ancestral CAS (AncCas) proteins using ASR includes performing phylogenetic tree reconstruction of Streptococcus Cas9 and ancestral Cas9 sequence and structure prediction.

In one embodiment, the phylogenetic tree is constructed using maximum likelihood and Bayesian analysis of aligned sequence data gathered from the NCBI database.

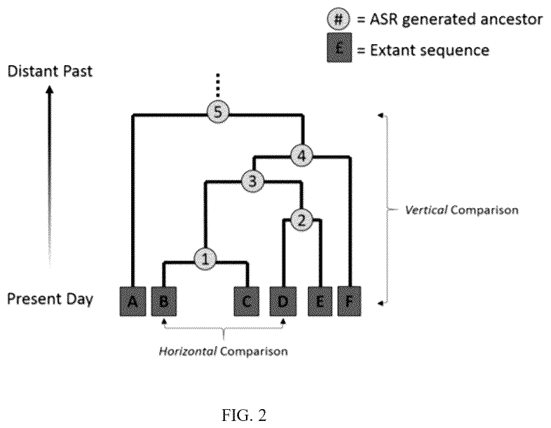

Referring to FIG. 2, demonstrates that in the described method for ASR of Cas proteins and CRISPR RNA, ASR can use currently known extant sequences to generate ASR generated ancestral sequences.

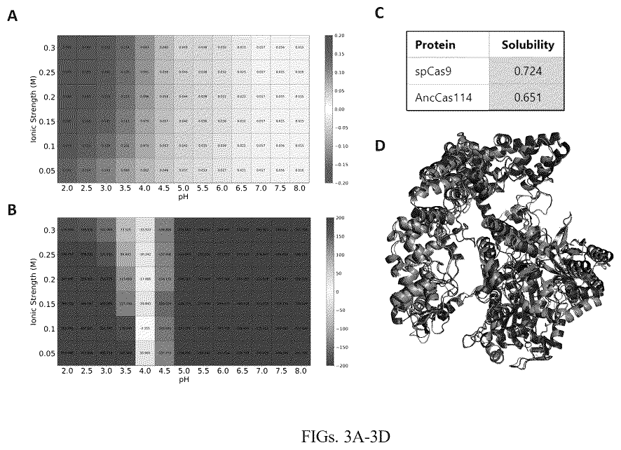

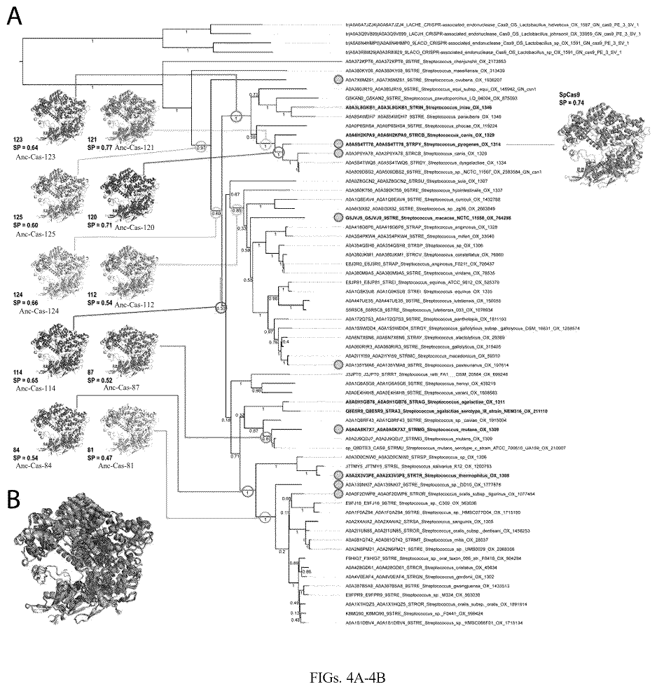

Turning now to FIGS. 3A-D, the method for identifying and creating novel ancestral CAS (AncCas) proteins using ASR includes performing computational analysis of the predicted AncCas sequences.

In one embodiment, the computational analysis includes heat maps showing the ionic strength of the resulting AncCas proteins over the pH (FIG. 3A and FIG. 3B).

In one embodiment, the computational analysis includes solubility predictions of the resulting AncCas proteins, for example the solubility of AncCas-114 compared to SpCas9 (FIG. 3C). The computational analysis can also include Alphafold generated structural predictions of the resulting AncCas proteins (FIG. 3D). FIG. 3D illustrates AncCas-114.

Referring now to FIG. 4A, the method for identifying and creating novel ancestral CAS (AncCas) proteins using ASR includes using phylogenetic and computational analysis methods to guide selection of Cas9 enzymes to use in the ASR analysis.

In one embodiment, the phylogenetic analysis includes protein sequences from Streptococcus, Lactobacillus, and Ancestral Cas protein sequences.

In one embodiment the Cas protein is Cas9 or any other suitable Cas enzyme.

In one embodiment, the phylogenetic analysis includes protein sequences from at least about 10, at least about 20, at least about 30, at least about 50, or at least about 60, at least about 70, at least about 80, or at least about 90 Streptococcus Cas protein sequences.

In one embodiment, the phylogenetic analysis includes protein sequences from only Streptococcus Cas protein sequences.

In one embodiment, the phylogenetic analysis includes protein sequences from at least about 2, at least about 4, at least about 10, at least about 20, or at least about 30 Lactobacillus Cas protein sequences.

In one embodiment, the phylogenetic analysis includes protein sequences at least 10, at least 20, at least 30, at least 50, or at least 60 Ancestral Cas protein sequences.

In one embodiment, the phylogenetic analysis includes at least about 62 Streptococcus, at least about 4 Lactobacillus, or at least about 61 Ancestral Cas9 protein sequences.

In one embodiment, the phylogenetic analysis includes using a protein amino acid sequence BLAST of SpCas9 was to gather about 66 Streptococcus Cas9 protein sequences, performing maximum-likelihood analysis using the MEGA X or any other suitable software and inferred ASR using ProtASR2 or any other suitable software known in the art. As a result, the phylogenetic analysis can produce 61 potential ancestral protein sequences with varying degrees of bootstrap value confidence.

In one embodiment, the method for identifying and creating novel ancestral CAS (AncCas) proteins using ASR includes using the phylogenetic and computational analysis methods wherein higher bootstrapping values (e.g., bootstrapping values of 20 or greater) are used to select ancestral candidates with a higher confidence that they will be functional.

In one embodiment, the selected AncCas protein can comprise a predicted solubility score (SP) value of at least about 0.2, or at least about 0.4, at least about 0.5, at least about 0.6, at least about 0.7, or at least about 0.8.

In one embodiment, the method for identifying and creating novel ancestral CAS (AncCas) proteins using ASR includes using several tools to screen for sequences that have a higher likelihood of being functional.

In one embodiment, several tools to screen for sequences that have a higher likelihood of being functional includes measuring predicted solubility, folding stability, and structural similarity to existing Cas proteins. For example, a root mean square deviation (RMSD) of less than about 5.0 angstroms when superimposing the known or predicted structures of an extant and ancestral Cas protein would be expected to increase the likelihood of function.

In one embodiment, the method uses selection criteria designed to identify highly functional proteins and use quantitative measurements of confidence including bootstrapping values (e.g., solubility, and folding/stability).

In one embodiment, the full sample set of potential ancestral sequences are analyzed using in-silico computational analysis programs (e.g., Alphafold2, SoluProt, Protein-Sol, and MEDUSA). The results from the computational analysis are compared to bootstrap confidence values along with distance to the root SpCas9 species and in one embodiment, twenty protein sequences are selected for further classification, ten extant proteins and ten ancestral proteins.

In one embodiment as shown in FIG. 4A, extant Cas9 (ExtCas) protein sequences chosen for characterization are indicated in red circles, with SpCas9 specifically identified along with its solubility score (SP) according to Soluprot or any other suitable software known in the art.

In one embodiment, the exemplary ancestral Cas9 (AncCas) sequences are identified in FIG. 4A at their place in the phylogenetic tree. SoluProt solubility scores are reported next to the ancestral protein structures indicating high degree of solubility prediction (FIG. 4A).

In one embodiment, the method for identifying and creating novel ancestral AncCas proteins using ASR can identify functional AncCas proteins comprising at least one of AncCas-123, AncCas-121, AncCas-125, AncCas-120, AncCas-124, Anc-Cas-112, Anc-Cas-114, AncCas-87, Anc-Cas-84, and Anc-Cas-81. AncCas-123, AncCas-121, AncCas-125, AncCas-120, AncCas-124, Anc-Cas-112, Anc-Cas-114, AncCas-87, Anc-Cas-84, and Anc-Cas-81 comprise amino acid sequences wherein the sequences have a sequence similarity of at least about 95% to the sequences SEQ. ID. NOs. 1-10 as identified in Table 1.

In one embodiment, the method for identifying and creating novel ancestral AncCas proteins comprises identifying AncCas proteins having an amino acid sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to a sequence selected from the group consisting of SEQ. ID. NOs. 1-10 and a functional fragment thereof.

In one embodiment, a composition is provided wherein the composition comprises at least one AncCas protein having a sequence wherein the sequence has a sequence similarity of at least about 25%, at least about 50%, at least about 70%, at least about 80%, at least about 90%, at least about 95%, at least about 96%, at least about 97%, at least about 98%, at least about 99% with respect to a sequence selected from the group consisting of SEQ. ID. NOs. 1-10 and functional fragments thereof.

In one embodiment, a composition is provided wherein the composition comprises at least one AncCas protein having a sequence wherein the sequence has a sequence similarity of at least 95%, at least 96%, at least 97%, at least 98%, or at least 99% with respect to at least one sequence selected from the group consisting of SEQ. ID. NOs. 1-10 and functional fragments thereof.

TABLE 1

| SEQ. | | |
|---|---|---|
| ID. NO. | AncCas protein | Amino Acid Sequence |
| 1 | AncCas-114 | MKKPYSIGLDIGTNSVGWAVITDDYKVPAKKMKVLGNTDKQYIKKNLLGALLFD |
| | | SGETAEARRLKRTARRRYTRRRNRIRYLQEIFAEEMNKVDESFFHRLDDSFLVPED |
| | | KRGERHPIFGNLAEEVAYHENFPTIYHLRKHLADSPEKADLRLVYLALAHIIKFRG |
| | | HFLIEGDLNSENTDVQKLFNEFVEVYDKTFEESHLSEQTVDVSAILTEKISKSRRLE |
| | | NLLKHFPNEKKNGLFGNFLALSLGLQPNFKSNFDLSEDAKLQFSKDTYEEDLENLL |
| | | GQIGDEYADLFVAAKNLYDAILLSGILTVNDNSTKAPLSASMVKRYEEHQEDLAQ |
| | | LKEFIRENLPDKYKEIFSDKSKNGYAGYIEGKTSQEDFYKYLKGILSKIDGSEYFLE |
| | | KIDREDFLRKQRTFDNGSIPHQIHLQEMHAILRRQGEYYPFLKENQEKIEKILTFRIP |
| | | YYVGPLARGNSRFAWLSRKSDEKITPWNFDEVVDKESSAEAFIERMTNYDLYLPD |
| | | EKVLPKHSLLYEKFTVYNELTKVKYVTEQGKAQFFDANMKQEIFDGLFKENRKV |
| | | TKKKLMDYLDKEFDEFRIVDLTGLDKENKAFNASLGTYHDLLKIIKDKDFLDNPE |
| | | NEDILEDIVHTLTLFEDREMIKQRLAKYSDLFDKKVLKKLARRHYTGWGRLSAKL |
| | | INGIRDKQSRKTILDYLIDDGYSNRNFMQLINDDSLSFKEEIAKAQVIGDTDDLKQV |
| | | VQDLAGSPAIKKGILQSIKIVDELVKVMGHNPENIVIEMARENQTTNRGRRNSQQR |
| | | LKRLEDAIKNLGSNILKEHPVDNQQLQNDRLFLYYLQNGKDMYTGEPLDIDRLSQ |
| | | YDIDHIIPQAFIKDDSIDNRVLTSSAKNRGKSDNVPSLEVVRKMKAFWQQLLNAKL |
| | | ISQRKFDNLTKAERGGLTEDDKAGFIKRQLVETRQITKHVARILDERFNTELDENG |
| | | KRIRKVKIITLKSNLVSQFRKDFELYKVREINDYHHAHDAYLNAVVGTALLKKYP |
| | | KLAPEFVYGDYPKYNSYKERGKATEKMFFYSNIMNFFKTEVKLADGTVVERPMIE |
| | | VNEETGEIVWDKEKDFATVRKVLSYPQVNIVKKTEVQTGGFSKESILPKGNSDKLI |
| | | PRKTKYWDPKKYGGFDSPTVAYSVLVVADVEKGKAKKLKTVKELVGITIMERSS |
| | | FEKNPVAFLEKKGYRNIQEDTIIKLPKYSLFELENGRRRLLASAGELQKGNEMVLP |
| | | QHLVTLLYHAQRINKTNEPEHLEYVEQHRHEFDELLNYIIDFAEKYVLAEKNLEKI |
| | | KELYSKNEDASIEELASSFINLLTFTALGAPAAFKFFGTNIPRKRYTSTTECLNATLI |
| | | HQSITGLYETRIDLSKLGED* |
| 2 | AncCas-81 | MKKPYSIGLDIGTNSVGWAVITDDYKVPAKKMKVLGNTDKHFIKKNLLGALLFD |
| | | SGTTAEDRRLKRTARRRYTRRRNRLRYLQEIFSEEMSKVDSSFFHRLDDSFLVPED |
| | | KRGSRYPIFGTLAEEKEYHENFPTIYHLRKHLADSKEKADLRLIYLALAHMIKYRG |
| | | HFLYEESFDSKNNDIQKIFNEFISIYDNTFEGSSLSGQNAQVEAILTDKISKSAKRERI |
| | | LKLFPDEKSTGLFSEFLKLIVGNQADFKKHFDLEEKAPLQFSKDTYDEDLENLLGQ |
| | | IGDDFADLFLVAKKLYDAILLSGILTVTDVSTKAPLSASMIERYENHQKDLAALKQ |
| | | FIKNNLPEKYNEVFSDQSKDGYAGYIDGKTTQEAFYKYIKNLLSKFEGADYFLEKI |
| | | EREDFLRKQRTFDNGSIPHQIHLQEMNAILRRQGEHYPFLKENKEKIEKILTFRIPY |
| | | YVGPLARGNRDFAWLTRNSDQAIRPWNFEEVVDKASSAEDFINKMTNYDLYLPE |
| | | EKVLPKHSLLYETFAVYNELTKVKFIAEGLRDYQFLDSGQKKQIVNLLFKEKRKV |
| | | TEKDIIHYLHNVDGYDGIELKGIDKQFNASLSTYHDLLKIIKDKDFMDDPENEAILE |
| | | NIVHTLTIFEDREMIKQRLAQYDSIFDKKVIKALTRRHYTGWGKLSAKLINGIRDK |
| | | QTGKTILDYLIDDGEINRNFMQLINDDSLSFKETIQKAQVIGETDNIKQVVQELPGS |
| | | PAIKKGILQSIKIVDELVKVMGHNPESIVVEMARENQTTNQGRRNSQQRLKRLEDS |
| | | LKNLGSNILKEHPVDNSQLQNDRLFLYYLQNGKDMYTGEALDINQLSSYDIDHIIP |
| | | QAFIKDDSLDNRVLVSSAKNRGKSDDVPSLEVVQKRKAFWQQLLDSKLISERKFN |
| | | NLTKAERGGLTEDDKAGFIKRQLVETRQITKHVAQILDARFNTEVTEKDKKNRTV |
| | | KIITLKSNLVSNFRKEFELYKVREINDYHHAHDAYLNAVVAKALLKKYPKLEPEF |
| | | VYGDYPKYNSYRERKKATEKVFFYSNIMNFFKTEVKLADGTVVKREQIEVNEETG |
| | | EIVWNKEKHFATVRKVLSYPQVNIVKKTEVQTVGQNGGLFDNNIVPKGKVVNAD |
| | | KLIPIKSGLWDPEKYGGYARPTIAYSVLVIADIEKGKAKKLKTVKEMVGITIMDKK |
| | | KFEKNPIAYLEEKGYKNINKNNIIKLPKYSLFEFENGRRRLLASSKELQKGNELVLP |
| | | YHLTTLLYHAKRINKISEPEHLEYVEKHRNEFEELLNTIIEFSKKYVLAKANVEKIK |
| | | QLYFDNEDADIEQLANSFISLLTFTSLGAPAAFKFFGVDISRSRYTSVSSCLNATLIH |
| | | QSITGLYETRIDLSKLGED* |
| 3 | AncCas-84 | MKKPYSIGLDIGTNSVGWAVITDDYKVPAKKMKVLGNTDKEYIKKNLLGALLFD |
| | | SGETAEDRRLKRTARRRYTRRRNRILYLQEIFSEEMSKVDDSFFHRLDDSFLVPED |
| | | KRGSRYPIFGNLAEEKAYHENFPTIYHLRKHLADSKEKADLRLVYLALAHMIKYR |
| | | GHFLIEGDFDSKNNDIQKIFQEFLEVYDNTFENSHLSEQNAQVEAILTDKISKSAKK |
| | | ERILKLFPNEKSTGLFSEFLKLIVGNQADFKKHFDLEEKAPLQFSKDTYDEDLENLL |
| | | GQIGDDYADLFLAAKKLYDAILLSGILTVTDVSTKAPLSASMIQRYEEHQEDLAQL |
| | | KQFIRNNLPEKYNEVFSDQSKNGYAGYIDGKTNQEAFYKYLKNLLSKFEGADYFL |
| | | EKIEREDFLRKQRTFDNGSIPHQIHLQEMRAILRRQGEYYPFLKENKEKIEKILTFRI |
| | | PYYVGPLARGNSDFAWLTRKSDEKITPWNFEEVVDKESSAEAFINRMTNYDLYLP |
| | | EEKVLPKHSLLYETFTVYNELTKVKFIAEGLRDYQFLDSNQKKEIVNLLFKEKRKV |
| | | TEKDIIDYLHKVDGYDGIELKGIDKQFNASLSTYHDLLKIIKDKDFLDDPENEAILE |
| | | DIVHTLTIFEDREMIKQRLAKYDDIFDKKVLKKLTRRHYTGWGKLSAKLINGIRDK |
| | | QSGKTILDYLIDDGNSNRNFMQLINDDALSFKEEIQKAQVIGDTDNIKQVVQDLPG |
| | | SPAIKKGILQSIKIVDELVKVMGHNPESIVVEMARENQTTNQGRRNSQQRLKRLED |
| | | SLKNLGSNILKEHPVDNNQLQNDRLFLYYLQNGKDMYTGEALDIDRLSSYDIDHII |
| | | PQAFIKDDSIDNRVLVSSAKNRGKSDDVPSLEVVKKRKAFWQQLLDSKLISQRKF |
| | | DNLTKAERGGLTEDDKAGFIKRQLVETRQITKHVARILDERFNTEVDENNKKIRTV |
| | | KIITLKSNLVSNFRKDFELYKVREINDYHHAHDAYLNAVVAKALLKKYPKLEPEF |
| | | VYGDYPKYNSYRERKKATEKVFFYSNIMNFFKKEVKLADGTVVERPMIEVNEET |
| | | GEIVWNKEKHFATVRKVLSYPQVNIVKKTEVQTGGFSKENILPKGKEVNSDKLIPR |
| | | KTKYWDPKKYGGFDSPTVAYSVLVIADIEKGKAKKLKTVKELVGITIMEKSKFEK |
| | | NPIAFLEEKGYRNIQEDNIIKLPKYSLFELENGRRRLLASAGELQKGNELVLPNHLV |
| | | TLLYHAKRINKTSEPEHLEYVEKHRNEFEELLNVIIEFSEKYILAEANLEKIKELYQ |
| | | KNENADIEELASSFINLLTFTSLGAPAAFKFFGVNIPRKRYTSVSECLNATLIHQSIT |
| | | GLYETRIDLSKLGED* |
| 4 | AncCas-87 | MKKPYSIGLDIGTNSVGWAVVTDDYKVPAKKMKVLGNTDKSHIKKNLLGALLFD |
| | | SGNTAEDRRLKRTARRRYTRRRNRILYLQEIFSEEMGKVDDSFFHRLEDSFLVTED |
| | | KRGERHPIFGNLEEEVKYHENFPTIYHLRQYLADNPEKVDLRLVYLALAHIIKFRG |
| | | HFLIEGKFDTRNNDVQRLFQEFLAVYDNTFENSSLQEQNVQVEEILTDKISKSAKK |
| | | DRVLKLFPNEKSNGRFAEFLKLIVGNQADFKKHFELEEKAPLQFSKDTYEEDLEVL |
| | | LAQIGDDYADLFLSAKKLYDSILLSGILTVTDVSTKAPLSASMIQRYNEHQMDLAQ |
| | | LKQFIRQKLSDKYNEVFSDVSKDGYAGYIDGKTNQEAFYKYLKGLLNKIEGSGYF |
| | | LDKIEREDFLRKQRTFDNGSIPHQIHLQEMRAIIRRQAEFYPFLADNQDRIEKILTFR |
| | | IPYYVGPLARGKSDFAWLSRKSADKITPWNFDEIVDKESSAEAFINRMTNYDLYLP |
| | | NQKVLPKHSLLYEKFTVYNELTKVKYKTEQGKTAFFDANMKQEIFDGVFKVYRK |
| | | VTKDKLMDFLEKEFDEFRIVDLTGLDKENKAFNASYGTYHDLRKILDKDFLDNSK |
| | | NEKILEDIVLTLTLFEDREMIRKRLENYSDLLTKEQVKKLERRHYTGWGRLSAELI |
| | | HGIRNKESRKTILDYLIDDGNSNRNFMQLINDDALSFKEEIAKAQVIGETDNLNQV |
| | | VSDIAGSPAIKKGILQSLKIVDELVKIMGHQPENIVVEMARENQFTNQGRRNSQQR |
| | | LKGLTDSIKEFGSQILKEHPVENSQLQNDRLFLYYLQNGRDMYTGEELDIDYLSQY |
| | | DIDHIIPQAFIKDNSIDNRVLTSSKENRGKSDDVPSKDVVRKMKPFWSKLLSAKLIT |
| | | QRKFDNLTKAERGGLTDDDKAGFIKRQLVETRQITKHVARILDERFNTELDENNK |
| | | KIRQVKIVTLKSNLVSNFRKEFELYKVREINDYHHAHDAYLNAVVGKALLGVYPQ |
| | | LEPEFVYGDYPHFHGHKEENKATAKKFFYSNIMNFFKKDVVRTDGTIVERPMIEV |
| | | NDENGEIIWKKDEHISNIKKVLSYPQVNIVKKVEEQTGGFSKESILPKGNSDKLIPR |
| | | KTKKFYWDTKKYGGFDSPIVAYSILVIADIEKGKSKKLKTVKALVGITIMEKMTFE |
| | | RNPVAFLERKGYRNIQEENIIKLPKYSLFELENGRKRLLASARELQKGNEIVLPNHL |
| | | GTLLYHAKNIHKVDEPKHLDYVDKHKDEFKELLDVVSNFSKKYTLAEGNLEKIKE |
| | | LYAQNNSEDIKELASSFINLLTFTAIGAPATFKFFDKNIDRKRYTSTTEILNATLIHQ |
| | | SITGLYETRIDLSKLGGD* |
| 5 | AncCas-112 | MKKPYSIGLDIGTNSVGWAVITDDYKVPAKKMKVLGNTDKQYIKKNLLGALLFD |
| | | SGETAEATRLKRTARRRYTRRRNRLRYLQEIFAEEMNKVDESFFHRLDESFLVPED |
| | | KRGERHPIFGNIAEEVAYHQKFPTIYHLRKHLADSSEKADLRLVYLALAHIIKFRG |
| | | HFLIEGDLNAENTDVQKLFNDFVEVYDKTVEESHLSEITVDAAAILTEKISKSRRLE |
| | | NLIKHYPTEKKNTLFGNLIALSLGLQPNFKTNFQLSEDAKLQFSKDTYEEDLEELL |
| | | GQIGDDYADLFVAAKNLYDAILLSGILTVNDNSTKAPLSASMVKRYEEHQEDLAQ |
| | | LKEFIKENAPDKYNEIFKDKSKNGYAGYIENKVKQEDFYKYLKGILSKIDGSEYFL |
| | | DKIDREDFLRKQRTFDNGSIPHQIHLQEMHAILRRQGEYYPFLKENQDKIEKILTFR |
| | | IPYYVGPLARKNSRFAWASYKSDEKITPWNFDEVVDKEKSAEKFITRMTSYDLYL |
| | | PEEKVLPKHSLVYEKFTVYNELTKVKYVNEQGKAKFFDANMKQEIFDHVFKENR |
| | | KVTKEKLLNYLNKEFEEFRIVDLTGLDKENKAFNASLGTYHDLKKILDKDFLDDK |
| | | ANEDIIEDIIHTLTLFEDREMIRQRLQKYSDLFTKKQLKKLERRHYTGWGRLSYKLI |
| | | NGIRNKETNKTILDYLIDDGYANRNFMQLINDDSLSFKEEIAKAQVIGDVDDLKQV |
| | | VHDLAGSPAIKKGILQSVKIVDELVKVMGHNPENIVIEMARENQTTNRGRRNSQQ |
| | | RLKRLQDSLKNLGSKILNEKNVENQQLQNDRLFLYYLQNGKDMYTGEPLDIDHLS |
| | | QYDIDHIIPQAFIKDDSIDNRVLTSSAKNRGKSDNVPSLEVVRKRKAYWQRLRKA |
| | | KLISQRKFDNLTKAERGGLTEDDKAGFIKRQLVETRQITKHVAQILDARFNTERDE |
| | | NGKVIRDVKIITLKSNLVSQFRKDFELYKVREINDYHHAHDAYLNAVVGTALLKK |
| | | YPKLAPEFVYGEYKKYNVRKLIAKERGKATAKKFFYSNLMNFFKTEVKYADGTV |
| | | VERPVIETNEETGEIVWNKEKDFATVRKVLSYPQVNIVKKVEVQTGGFSKESILPK |
| | | GDSDKLIPRKTKNVYWDPKKYGGFDSPTVAYSVLVVADVEKGKAKKLKTVKEL |
| | | VGISIMERSAFEKNPVAFLEKKGYRNIQEDTIIKLPKYSLFELENGRRRLLASAGEL |
| | | QKGNEMVLPQKLVTLLYHAHRINNSNEPEHLEYVEKHKEEFKELLNYIVEFAEKY |
| | | VLAEKNLEKIQALYSKNDDASIEELASSFINLLTFTALGAPAAFKFLGAKIPRKRYT |
| | | STTECLNATLIHQSITGLYETRIDLSKLGED* |
| 6 | AncCas-120 | MDKKYSIGLDIGTNSVGWAVITDDYKVPSKKFKVLGNTDRHSIKKNLIGALLFDS |
| | | GETAEATRLKRTARRRYTRRKNRIRYLQEIFSNEMAKVDDSFFHRLEESFLVEEDK |
| | | KHERHPIFGNIVDEVAYHEKYPTIYHLRKKLADSTDKADLRLIYLALAHMIKFRGH |
| | | FLIEGDLNPDNSDVDKLFIQLVQTYNQLFEENPINASRVDAKAILSARLSKSRRLEN |
| | | LIAQLPGEKKNGLFGNLIALSLGLTPNFKSNFDLAEDAKLQLSKDTYDDDLDNLLA |
| | | QIGDQYADLFLAAKNLSDAILLSDILRVNSEITKAPLSASMIKRYDEHHQDLTLLK |
| | | ALVRQQLPEKYKEIFFDQSKNGYAGYIDGGASQEEFYKFIKPILEKMDGTEELLAK |
| | | LNREDLLRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFDKNLPNE |
| | | KVLPKHSLLYEYFTVYNELTKVKYVTEGMRKPAFLSGEQKKAIVDLLFKTNRKVT |
| | | VKQLKEDYFKKIECFDSVEISGVEDRFNASLGTYHDLLKIIKDKDFLDNEENEDILE |
| | | DIVLTLTLFEDREMIEERLKTYAHLFDDKVMKQLKRRHYTGWGRLSRKLINGIRD |
| | | KQSGKTILDFLKSDGFANRNFMQLIHDDSLTFKEEIQKAQVSGQGDSLHEQIANLA |
| | | GSPAIKKGILQTVKVVDELVKVMGHKPENIVIEMARENQTTQKGQKNSRERMKRI |
| | | EEGIKELGSQILKEHPVENTQLQNEKLYLYYLQNGRDMYVDQELDINRLSDYDVD |
| | | HIVPQSFIKDDSIDNKVLTRSDKNRGKSDNVPSEEVVKKMKNYWRQLLNAKLITQ |
| | | RKFDNLTKAERGGLSELDKAGFIKRQLVETRQITKHVAQILDSRMNTKYDENDKL |
| | | IREVKVITLKSKLVSDFRKDFQFYKVREINNYHHAHDAYLNAVVGTALIKKYPKL |
| | | ESEFVYGDYKVYDVRKMIAKSEQEIGKATAKRFFYSNIMNFFKTEITLANGEIRKR |
| | | PLIETNGETGEIVWDKGRDFATVRKVLSMPQVNIVKKTEVQTGGFSKESILSKGNS |
| | | DKLIARKKDWDPKKYGGFGSPTVAYSVLVVAKVEKGKAKKLKSVKELIGITIMER |
| | | SSFEKDPIDFLEAKGYKDVQKDLIIKLPKYSLFELENGRRRMLASAGELQKGNEM |
| | | VLPSKLVTFLYHASHIEKSKGSQHQAYVEQHRHDLDEILEYISEFSKRYILADKNLS |
| | | KVKSAFNKHEDYSISELASSIINLFTLTSLGAPAAFKFLDTTIDRKRYTSTKEVLDAT |
| | | LIHQSITGLYETRIDLSQLGGD* |
| 7 | AncCas-121 | MEKKYSIGLDIGTNSVGWAVITDDYKVPSKKFKVLGNTDRQSIKKNLIGALLFDS |
| | | GETAEATRLKRTARRRYTRRKNRIRYLQEIFANEMAKVDDSFFHRLEESFLVEED |
| | | KKHERHPIFGNLADEVAYHENYPTIYHLRKKLADSPEKADLRLIYLALAHIIKFRG |
| | | HFLIEGDLNAENSDVDKLFYQLVQTYNQLFEENPLDASRVDAKAILSARLSKSRRL |
| | | ENLIAQLPGEKKNGLFGNLIALSLGLTPNFKSNFDLAEDAKLQLSKDTYDDDLDNL |
| | | LAQIGDQYADLFLAAKNLSDAILLSDILRVNSEITKAPLSASMVKRYDEHHQDLAL |
| | | LKALVRQQLPEKYKEIFSDQSKNGYAGYIDGKASQEEFYKFIKPILEKMDGAEELL |
| | | AKLNREDLLRKQRTFDNGSIPHQIHLGELHAILRRQEEFYPFLKDNREKIEKILTFRI |
| | | PYYVGPLARGNSRFAWLTRKSEETITPWNFEEVVDKGASAQSFIERMTNFDENLP |
| | | NEKVLPKHSLLYEYFTVYNELTKVKYVTEGMRKPEFLSGEQKKAIVDLLFKTNRK |
| | | VTVKQLKEDYFKKIECFDSVEISGVEDRFNASLGTYHDLLKIIKDKDFLDNEENEDI |
| | | LEDIVLTLTLFEDREMIEERLKTYAHLFDDKVMKQLKRRHYTGWGRLSRKLINGI |
| | | RDKQSGKTILDFLKSDGFANRNFMQLIHDDSLTFKEEIQKAQVSGQGDSLHEQIAN |
| | | LAGSPAIKKGILQTVKIVDELVKVMGHKPENIVIEMARENQTTQKGLKNSRERMK |
| | | RIEEGIKELGSQILKEHPVENTQLQNEKLYLYYLQNGRDMYVDQELDINRLSDYD |
| | | VDHIVPQSFIKDDSIDNKVLTSSDKNRGKSDNVPSEEVVKKMKSYWRQLLNAKLI |
| | | SQRKFDNLTKAERGGLSEADKAGFIKRQLVETRQITKHVAQILDSRMNTKYDEND |
| | | KPIREVKVITLKSKLVSDFRKDFQFYKVREINNYHHAHDAYLNAVVGTALIKKYP |
| | | KLESEFVYGDYKVYDVRKMIAKSEQEIGKATAKRFFYSNIMNFFKTEVKLANGEI |
| | | RKRPLIETNGETGEIVWDKEKDFATVRKVLSMPQVNIVKKTEVQTGGFSKESILSK |
| | | GNSDKLIPRKKNWDPKKYGGFGSPTVAYSVLVVAKVEKGKAKKLKSVKELVGIT |
| | | IMERSSFEKDPIAFLEAKGYKDIQKDLIIKLPKYSLFELENGRRRMLASAGELQKGN |
| | | EMVLPQHLVTFLYHASHIEKTTGSENLAYIEQHRHEFDEILEYIIEFSERYILADKNL |
| | | SKVKSAFNKHEDFSISELASSFINLFTFTSLGAPAAFKFLDTTIDQGRKRYTSTTEVL |
| | | DATLIHQSITGLYETRIDLSQLGGD* |
| 8 | AncCas-123 | MKKPYSIGLDIGTNSVGWAVITDDYKVPAKKMKVLGNTDRQSIKKNLIGALLFDS |
| | | GETAEATRLKRTARRRYTRRKNRIRYLQEIFAEEMAKVDDSFFQRLEDSFLVPEDK |
| | | KYERHPIFGNLADEVAYHENYPTIYHLRKKLADSPEKADLRLIYLALAHIIKFRGH |
| | | FLIEGDLNSENTDVQKLFHQLVDTYNLLFEEDPLDTETVDAKAILTAKISKSRRLE |
| | | NLIAQIPGQKKNGLFGNLIALSLGLTPNFKSNFDLSEDAKLQLSKDTYEEDLDNLL |
| | | AQIGDQYADLFLAAKNLSDAILLSDILTVNDESTKAPLSASMVKRYEEHQQDLAL |
| | | LKKLVREQLPEKYKEIFSDKSKNGYAGYIDGKTSQEEFYKYIKPILSKLDGAEEFL |
| | | AKIDREDFLRKQRTFDNGSIPHQIHLQELHAILRRQEEYYPFLKDNQEKIEKILTFRI |
| | | PYYVGPLARGNSRFAWLTRKSDEAITPWNFEEVVDKEASAQAFIERMTNFDLYLP |
| | | NEKVLPKHSLLYEMFTVYNELTKVKYVTEGMTKPEFLSAEQKQAIVDLLFKTNRK |
| | | VTVKQLKENYFKKIECFDSVDITGVEDRFNASLGTYHDLLKIIKDKDFLDNPENED |
| | | ILEDIVLTLTLFEDREMIEKRLAKYADLFDKKVLKKLKRRHYTGWGRLSRKLINGI |
| | | RDKQSGKTILDFLKADGFANRNFMQLINDDSLSFKEEIAKAQVIGQSDSLKEQVAN |
| | | LAGSPAIKKGILQTIKIVDELVKVMGHNPENIVIEMARENQTTAQGIKNSRQRMKR |
| | | LEEVIKKLGSQILKEHPVENTQLQNDRLYLYYLQNGKDMYTGEELDIDNLSQYDI |
| | | DHIIPQSFIKDDSIDNKVLTSSEKNRGKSDNVPSIEVVKKMKSYWQQLLNAKLISQ |
| | | RKFDNLTKAERGGLSESDKAGFIKRQLVETRQITKHVAQILDSRFNTELDENDKRI |
| | | RKVKIITLKSKLVSDFRKDFGLYKVREINDYHHAHDAYLNAVVGTALLKKYPKLA |
| | | PEFVYGDYKKYDLYKMIAKSDSERGKATAKMFFYSNIMNFFKTEVKLADGTIIKR |
| | | PLIEVNEETGEIVWDKEKDFATVRKVLSYPQVNIVKKTEVQTGGFSKESILSKGNS |
| | | DKLIPRKNNYWDPKKYGGFDSPTVAYSVLVVAKVEKGKAKKLKTVKELVGITIM |
| | | ERSSFEKNPIAFLEAKGYRDIQEHLIIKLPKYSLFELENGRRRLLASAGELQKGNEM |
| | | VLPQHLVTFLYHASRIDKLTGSEHLEYVEQHRHEFDEILEYIIEFAERYILADKNLE |
| | | KIKSTYNKKEDFSINELASSFINLFTFTALGAPAAFKFFDTTIDRKRYTSTTECLNAT |
| | | LIHQSITGLYETRIDLSQLGGD* |
| 9 | AncCas-124 | MKKPYSIGLDIGTNSVGWAVITDDYKVPAKKMKVLGNTDKQYIKKNLIGALLFDS |
| | | GETAEARRLKRTARRRYTRRRNRIRYLQEIFAEEMNKVDESFFHRLDDSFLVPEDK |
| | | RYERHPIFGNLAEEVAYHENFPTIYHLRKHLADSPEKADLRLVYLALAHIIKFRGH |
| | | FLIEGDLNSENTDVQKLFNEFVEVYDKLFEESHLSEQTVDVKAILTEKISKSRRLEN |
| | | LLAHFPNEKKNGLFGNFLALSLGLQPNFKSNFDLSEDAKLQFSKDTYEEDLENLLG |
| | | QIGDEYADLFLAAKNLYDAILLSGILTVNDESTKAPLSASMVKRYEEHQEDLAQL |
| | | KEFIRENLPDKYKEIFSDKSKNGYAGYIEGKTSQEDFYKYLKGILSKIDGSEYFLEK |
| | | IDREDFLRKQRTFDNGSIPHQIHLQELHAILRRQGEYYPFLKENQEKIEKILTFRIPY |
| | | YVGPLARGNSRFAWLSRKSDEKITPWNFDEVVDKEASAEAFIERMTNYDLYLPDE |
| | | KVLPKHSLLYEKFTVYNELTKVKYVTEGQGKPQFFSANMKQEIFDGLFKENRKVT |
| | | KKKLMDYLDKEFDEFRIVDITGLDKENKAFNASLGTYHDLLKIIKDKDFLDNPENE |
| | | DILEDIVHTLTLFEDREMIKQRLAKYSDLFDKKVLKKLARRHYTGWGRLSRKLIN |
| | | GIRDKQSRKTILDYLIDDGYSNRNFMQLINDDSLSFKEEIAKAQVIGDTDDLKQVV |
| | | ADLAGSPAIKKGILQSIKIVDELVKVMGHNPENIVIEMARENQTTNRGRRNSQQRL |
| | | KRLEDAIKNLGSNILKEHPVDNQQLQNDRLYLYYLQNGKDMYTGEPLDIDRLSQY |
| | | DIDHIIPQSFIKDDSIDNRVLTSSAKNRGKSDNVPSLEVVRKMKAFWQQLLNAKLIS |
| | | QRKFDNLTKAERGGLTEDDKAGFIKRQLVETRQITKHVARILDERFNTELDENGK |
| | | RIRKVKIITLKSNLVSQFRKDFGLYKVREINDYHHAHDAYLNAVVGTALLKKYPK |
| | | LAPEFVYGDYPKYNSYKERGKATEKMFFYSNIMNFFKTEVKLADGTVVERPMIEV |
| | | NEETGEIVWDKEKDFATVRKVLSYPQVNIVKKTEVQTGGFSKESILPKGNSDKLIP |
| | | RKTKYWDPKKYGGFDSPTVAYSVLVVADVEKGKAKKLKTVKELVGITIMERSSF |
| | | EKNPVAFLEKKGYRNIQEDTIIKLPKYSLFELENGRRRLLASAGELQKGNEMVLPQ |
| | | HLVTLLYHAQRINKTNEPEHLEYVEQHRHEFDELLDYIIDFAEKYVLAEKNLEKIK |
| | | ELYNKNEDASIEELASSFINLLTFTALGAPAAFKFFGTTIPRKRYTSTTECLNATLIH |
| | | QSITGLYETRIDLSKLGED* |
| 10 | AncCas-125 | MKKPYSIGLDIGTNSVGWAVITDDYKVPAKKMKVLGNTDKQYIKKNLIGALLFDS |
| | | GETAEARRLKRTARRRYTRRRNRIRYLQEIFAEEMNKVDESFFHRLDDSFLVPEDK |
| | | RYDRHPIFGTLAEEVAYHENFPTIYHLRKHLADSPEKADLRLVYLALAHIIKFRGH |
| | | FLIEGDLNSENTDVQKLFQEFVEVYDKLFEESHLSEQTVDVKAILTEKISKSRRVEN |
| | | ILALFPGLNEKKKGLFGQFLKLSLGLQANFKSIFDLSEDAKLQFSKDSYEEDLENLL |
| | | GQIGDEYADLFLAAKNLYDAILLSGILTVNDESTKAPLSASMVKRYEEHQEDLAQ |
| | | LKEFIRENLPDKYKEIFSDKSKNGYAGYIEGKTSQEDFYKYLKGILSKVEGSEYFLE |
| | | KIEREDFLRKQRTFDNGSIPHQIHLQELHAILRRQGKYYPFLKENKEKIEKILTFRIP |
| | | YYVGPLARGNSRFAWLSRKSDEKIRPWNFDEVVDKEASAEAFIERMTNHDLYLP |
| | | DEKVLPKHSLLYEKFTVYNELTKVRYVTEGQGKPQFFSANMKQEIFDHLFKEHRK |
| | | VTKKKLMDYLDKEFDEFRIVDITGLDKENKAFNASLSTYHDLKKIIKDKDFLDNPE |
| | | NQDILEEIVHTLTVFEDREMIKQRLAKYSDLFDKKVLKKLARRHYTGWGRLSRKL |
| | | INGIRDKQSRKTILDYLIDDGYSNRNFMQLINDDGLSFKEEIAKAQVIGDSDDLKQ |
| | | VVADLAGSPAIKKGILQSIKIVDELVKVMGYAPENIVIEMARENQTTNRGRRNSQQ |
| | | RLKRLEDAIKNLGSNILKEHPVDNQQLQNDRLYLYYLQNGKDMYTGEPLDIDRLS |
| | | QYDIDHIIPQSFIKDDSIDNRVLTSSAKNRGKLDNVPSLEVVKKMKAFWQQLYNA |
| | | KLISQRKFDNLTKAERGGLTEDDKAGFIKRQLVETRQITKHVARLLDERFNTELDE |
| | | NGKRIRTVKIITLKSNLVSQFRKDFGLYKVREINDYHHAHDAYLNAVVGKALLKK |
| | | YPKLAPEFVYGEYPKYNSYKERGKATEKMFFYSNIMNFFKKEVKLADGTVVERP |
| | | MIEVNEETGEIVWDKEKDFATVRKVLSYPQVNIVKKTEVQTGGFSKESILPKGNSD |
| | | KLIPRKTKYWDPKKYGGFDSPTVAYSVLVVADVEKGKAKKLKTVKELVGITIME |
| | | RSSFEKNPVAFLEKKGYRNIQEHTIIKLPKYSLFELENGRRRLLASAGELQKGNQM |
| | | VLPQHLVTLLYHAQRINKTNEPEHLEYVEQHRHEFDELLDYIVEFAEKYVLAEKN |
| | | LEKIKELYNKNEDASIEELASSFINLLTFTALGAPAAFKFFGTTIPRKRYTSTTECLN |
| | | ATLIHQSITGLYETRIDLSKLGED* |

Furthermore, in FIGS. 4A and 4B, exemplary Alphafold structural predictions (4B) are illustrated for exemplary AncCas proteins along with the predicted SP according to Soluprot or any other suitable software known in the art (4A). The predicted structures for the AncCas proteins show closely related protein folding to SpCas9 (FIG. 4B) confirming proper prediction methods and other computational analyses can infer functional derivatives of Cas9 enzymes.

In one embodiment, the AncCas protein can comprise a predicted SP of at least about 0.2, at least about 0.4, at least about 0.5, at least about 0.6, at least about 0.7, or at least about 0.8.

In one embodiment, the AncCas protein can comprise an amino acid sequence wherein the sequence has a sequence similarity of at least about 95%, at least about 96%, at least about 97%, at least about 98%, at least about 99% with respect to at least one sequence selected from the group consisting of SEQ. ID. NOs. 1-10 and functional fragments thereof and comprises a predicted SP of about 0.2 to about 1.0 or about 0.4 to about 0.8.

In one embodiment, the AncCas protein can comprise an amino acid sequence wherein the sequence has a sequence similarity of at least about 95% at least about 96%, at least about 97%, at least about 98%, or at least about 99% with respect to at least one sequence selected from the group consisting of SEQ. ID. NOs. 1-10 and functional fragments thereof and comprises a predicted SP of 0.2 to 1.0 or 0.4 to 0.8.

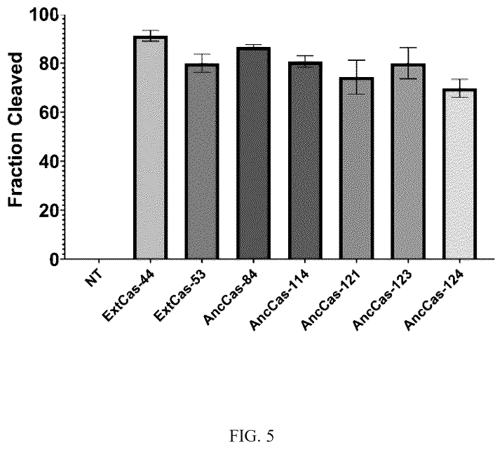

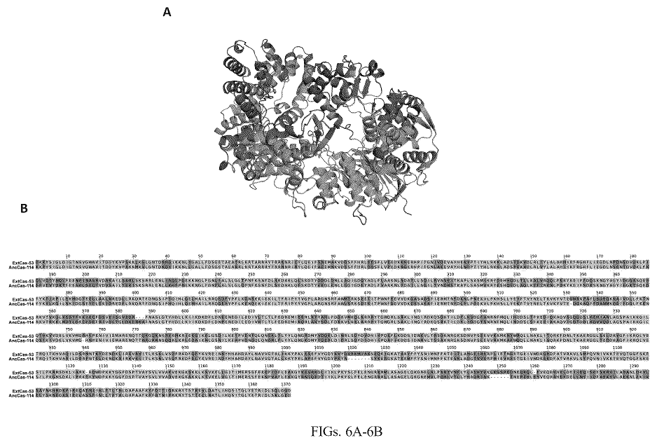

Turning now to FIG. 5, In one embodiment, the method for identifying and creating novel ancestral CAS (AncCas) proteins comprises in vitro biochemical assays including assessment of cleavage activity of the AncCas proteins to evaluate dsDNA cleavage efficiency. FIG. 5 shows the in vitro cleavage of dsDNA in the presence of AncCas and ExCas proteins (e.g., ExtCas-44 from Streptococcus thermophilus and ExtCas-53 from Streptococcus pyogenes). FIG. 5 presents in vitro cleavage assay efficiency for ExtCas and AncCas enzymes using guide RNA (e.g., sgRNA) from SpCas9. The results indicate that enzymatic activity is maintained when predicting ancestral Cas proteins.

In one embodiment, administration of the exemplary AncCas proteins can result in cleavage of dsDNA which can comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% fraction cleaved compared to the control lacking the Cas protein.

In one embodiment, various ancestral Streptococcus Cas9 enzymes (e.g., AncCas-84, AncCas-114, AncCas-121, AncCas-123, and AncCas-124) can efficiently cleave dsDNA.

Referring now to FIG. 6, In one embodiment, the resulting exemplary AncCas proteins can have an amount of structural deviation and sequence diversity from SpCas9. FIG. 6A illustrates an alignment of ExCas-53 and AncCas-114 with structurally similar residues colored green and structural changes colored red, indicating that many changes can occur on the protein surface of Cas9.

In one embodiment, the protein structure of AncCas-114 is significantly diverged from that of ExtCas-53 (SpCas9). In some instances, when superimposing the known structure of ExtCas-53 (SpCas9) and the predicted structure of AncCas-114, the root mean square deviation (RMSD) is less than about 5.0 angstroms.

In one embodiment, the protein structure of ExCas and AncCas proteins are determined using Alphafold or any other suitable software known in the art.

In one embodiment and as shown in FIG. 6B, AncCas proteins can exhibit at least about 20%, at least about 30%, at least about 40%, at least about 50%, at least about 60%, at least about 70%, at least about 80%, or at least about 90% sequence similarity to ExtCas-53 (SpCas9).

In one embodiment, the AncCas proteins can comprise an amino acid sequence. In such examples, the AncCas protein comprises the amino acid sequence having a sequence similarity of at least about 20% to about 90% to ExtCas-53 (SpCas9).

For example, the sequence similarity to ExtCas-53 (SpCas9) may be of at least about 20% at least about 30%, at least about 40%, at least about 50%, at least about 60%, at least about 70%, at least about 80%, or at least about 90%.

Typically, the sequence similarity to ExtCas-53 (SpCas9) is not greater than about 90%, not greater than about 80%, not greater than about 70%, not greater than about 60%, not greater than about 50%, not greater than about 40%, or not greater than about 30%.

The sequence similarity to SpCas9 may fall within a range bounded by any minimum value and any maximum value as described above. As a non-limiting example, the sequence similarity to ExtCas-53 (SpCas9) may fall within a range of from about 30% to about 90%.

In one embodiment, AncCas proteins can exhibit about 20%, about 30%, about 40%, about 50%, about 60%, about 70%, about 80%, or about 90% sequence similarity to ExtCas-53 (SpCas9).

In one embodiment, AncCas proteins can exhibit 20%, 30%, 40%, 50%, 60%, 70%, 80%, or 90% sequence similarity to ExtCas-53 (SpCas9).

In one embodiment, AncCas proteins can exhibit at least about 70% sequence similarity to ExtCas-53 (SpCas9).

In one embodiment, AncCas proteins can exhibit about 70% sequence similarity to ExtCas-53 (SpCas9).

In one embodiment, AncCas proteins can exhibit 70% sequence similarity to ExtCas-53 (SpCas9).

For example, FIG. 6B shows a sequence alignment of ExtCas-53 and AncCas-114 across the enzyme coding sequence, exhibiting about 70% sequence similarity between the sequences.

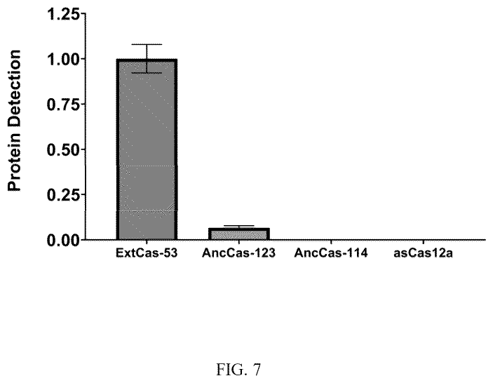

Turning now to FIG. 7, In one embodiment the various AncCas enzymes can deviate in sequence similarity from SpCas9, which can result in the AncCas proteins avoiding detection by anti-Cas9 antibodies. FIG. 7 shows that AncCas-123, AncCas-114 are not substantially detected by Anti-Cas9 antibody. In FIG. 7, ExtCas-53 was used as a positive control for the antibody binding assay where anti-Cas9 is expected to bind and be visualized by Western Blot antibody binding methods. Exemplary proteins AncCas-123 and AncCas-114 were used to represent different positions from the phylogenetic tree analysis with AncCas-123 being more closely related to ExtCas-53. In addition, purified AsCas12a protein was used as a negative control in which anti-Cas9 antibody is not expected to bind. As shown in FIG. 7, ImageJ quantification of normalized Western Blot detection data shows strong detection of ExtCas-53 and substantially less detection of AncCas-123, while AncCas-114 and AsCas12a are not detected.

In one embodiment, the AncCas proteins are not substantially detectable by Anti-Cas9 antibodies. In the context of ImageJ quantified normalized Western Blot detection data, substantially detectable can mean that the protein is not detected by more than greater than 0.025, 0.15, 0.10, or 0.05.

In one embodiment, various AncCas enzymes can deviate in sequence similarity from SpCas9, which can result in the AncCas proteins not being immunogenic or causing an immune response.

In one embodiment, antibody detection of anti-Cas9 is determined using an ELISA or any other suitable method known in the art.

In one embodiment, the ELISA can use commercially available antibodies against the reference Cas or human serum samples possessing human anti-Cas9 antibodies.

In one embodiment, a direct ELISA method using His-tagged Cas proteins bound to 96-well Ni-NT A coated plates with fluorescent secondary antibodies is used to measure the degree of antibody recognition.

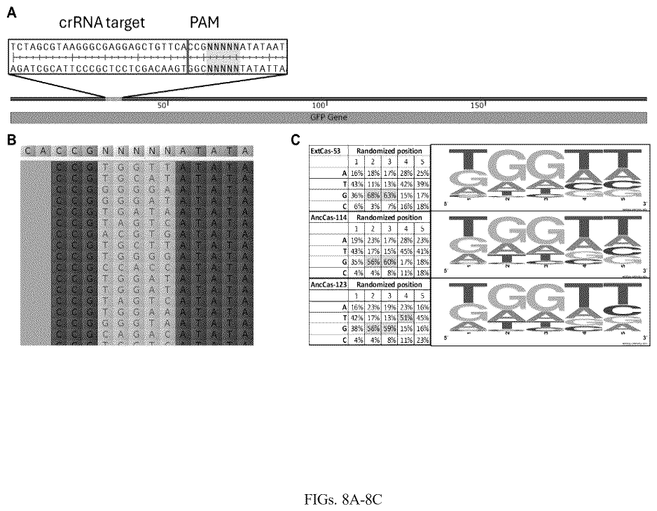

Referring now to FIG. 8, previous studies have explored the diversity of the Protospacer Adjacent Motif (PAM) in different Cas9 expressing species.

In one embodiment, the method for identifying and creating novel ancestral AncCas proteins comprises performing preliminary PAM finding assays.

In one embodiment, the preliminary PAM finding assays are performed for various AncCas and ExtCas enzymes. Purified AncCas proteins are assembled with candidate guide RNAs and combined with a target DNA containing a 5-nucleotide randomized PAM sequence (FIG. 8A).

In one embodiment, the design of PAM finding assay is illustrated by a 200-nucleotide target sequence with 5 randomized nucleotides upstream of a crRNA target cut site (FIG. 8A).

In one embodiment, following in vitro cleavage of the PAM-finding DNA target, cleaved DNA is sequenced by next-generation nanopore sequencing to identify DNA strands with target preferences in the nucleotide region and can determine PAM sequence preferences of the AncCas proteins. FIG. 4B illustrates representative sequencing results presenting reference sequence with randomized nucleotides (N) and aligned reads using PAM preference nucleotides. Representative sequencing results shown in FIG. 8B show sequenced strands with a double stranded break 3 nucleotides upstream of the randomized PAM sequence that were used for analysis. FIG. 8C demonstrates the results of the exemplary sequencing for ExtCas-53, AncCas-114, and AncCas-123.

In one embodiment, ExtCas-53 can exhibit the canonical NGGNN PAM preference, validating the assay. In one embodiment, AncCas-123 is closely related to ExtCas-53 and exhibit a preference for a NGGTN PAM preference with a >50% preference for thymine in the fourth nucleotide position compared to ExtCas-53.

In one embodiment, AncCas-114 sequencing can exhibit a more relaxed PAM preference compared to ExtCas-53 but maintained the NGGNN sequence. Therefore, in one embodiment, the PAM preference of Cas proteins including AncCas proteins is conserved across evolution of Cas9 in the Streptococcus genus.

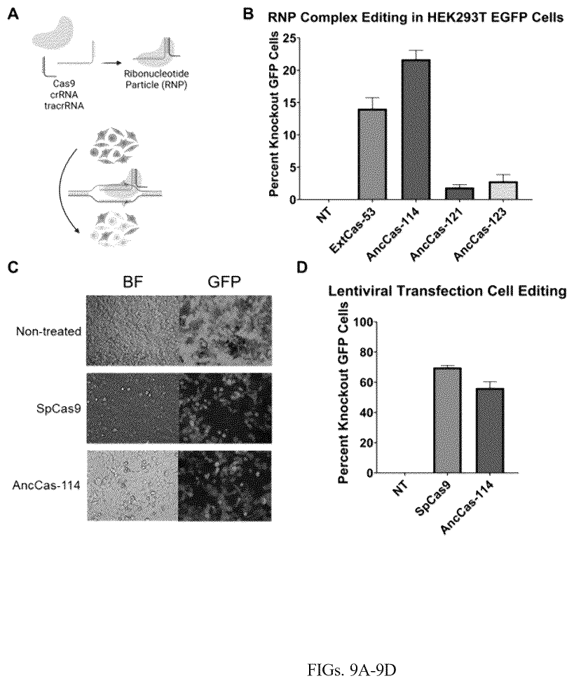

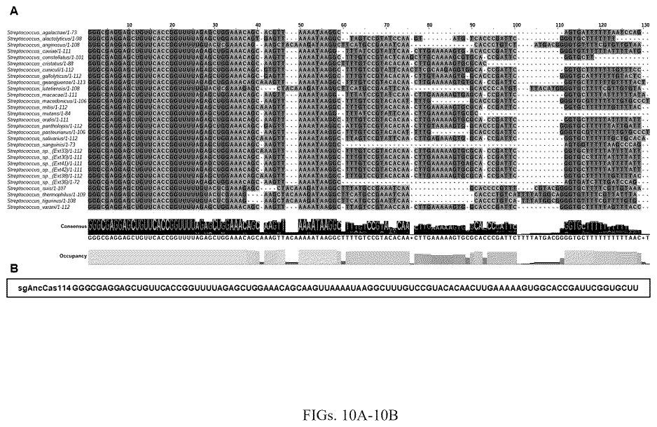

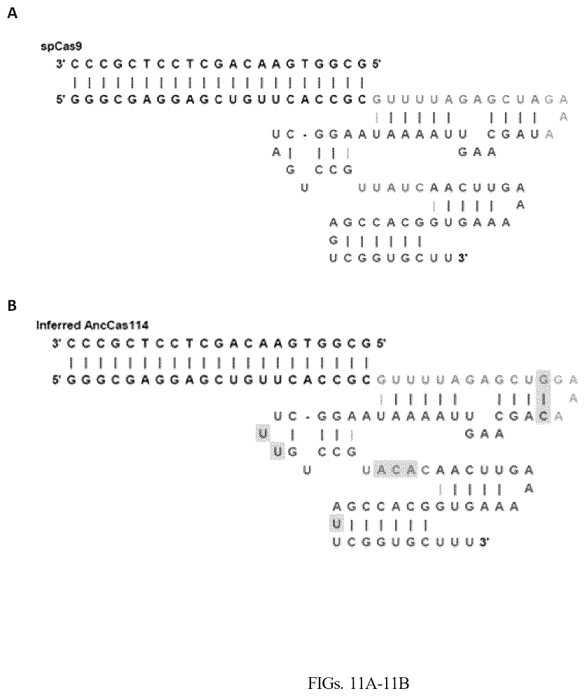

Turning now to FIG. 9, exemplary AncCas9 proteins can cleave and knockout target genes in human cell model systems, for example in HEK293T cells.

In one embodiment, the method for identifying and creating novel ancestral AncCas proteins comprises in vivo cell-based assays wherein the cell-based assay includes measurement of cell-based editing.

In one embodiment, cell-based editing or RNA editing assays is used.

Cell-based editing assays includes RNP assembly and transfection into HEK293T cells stably expressing green fluorescence protein (GFP) from an integrated GFP gene. FIG. 9A illustrates the targeted gene knockout experiment where the RNP was assembled in vitro and electroporated into model HEK cells. Following targeted cleavage of the GFP gene, cells can cease to produce fluorescent protein and are quantified by flow cytometry.

In one embodiment, the utility of the various AncCas enzymes in therapeutics or biomedical research is determined by measuring the capability of AncCas proteins for cell-based editing.

In one embodiment and as shown in FIG. 9B, ExtCas-53, AncCas-114, AncCas-121, and AncCas-123 can exhibit RNP complex editing.

In one embodiment AncCas-114 can exhibit increased knockout of GFP expressing cells when compared to the positive ExtCas-53 (SpCas9) control.

In one embodiment, measurement of cell-based editing or RNA editing assays can use AncCas with gRNAs targeting a model EGFP gene expressed constitutively in HEK293T cells, as described in Ageely, E. A et al. Gene editing with CRISPR-Cas12a guides possessing ribose-modified pseudoknot handles. Nature communications 12, 6591, and Barkau, C. L., O'Reilly, D., Rohilla, K. J., Damha, M. J. & Gagnon, K. T. Rationally Designed Anti-CRISPR Nucleic Acid Inhibitors of CRISPR-Cas9. Nucleic acid therapeutics 29, 136-147, the complete disclosures of which are hereby incorporated by reference in their entirety.

In one embodiment, cell-based editing is quantified by the loss of EGFP fluorescence measured using flow cytometry, a fluorescent plate reader, or any other suitable fluorescent reader, as a loss of EGFP signal.

In one embodiment, TIDE analysis, a Sanger sequencing-based method, is employed to accurately quantify EGFP gene editing.

In one embodiment, RNA degradation is measured. RNA degradation is measured by assaying endogenous transcripts using any suitable RT-qPCR methods.

In one embodiment and as shown in FIG. 9B, various AncCas proteins can cleave and knockout target genes in human cell model systems. Various AncCas proteins including AncCas-114, AncCas-121 and AncCas-123 can knockout GFP in in HEK293T cells as measured by flow cytometry.

In one embodiment, the AncCas protein can exhibit greater RNP complex editing in HEK293T cells compared to ExtCas-53 (SpCas9).

In one embodiment, AncCas-114 can exhibit greater RNP complex editing in HEK293T cells compared to ExtCas-53 (SpCas9).

In one embodiment, AncCas-114 is subcloned into a custom pLJM1 mammalian expression vector. Cloning success and sequence integrity is verified by nanopore sequencing.

In one embodiment, the mammalian expression vector and GFP targeting guide RNA (e.g., sgRNA) is transfected into cells and allowed to incubate. FIG. 9C shows that AncCas proteins including AncCas-114, (along with the SpCas9 control) can knockout GFP in in HEK293T cells as analyzed by microscopy using representative bright field (BF) and green fluorescence (GFP) microscope images of lentiviral expressed Cas9 enzymes and targeted GFP gene knockout. FIG. 5C shows representative microscopy images from targeted gene knockout using a lentiviral vector expressing SpCas9 and pLJM1 AncCas-114.

FIG. 9D presents a quantified analysis using flow cytometry to illustrate the results of lentiviral expressed Cas9 enzyme GFP KO. The results indicate that AncCas proteins (e.g., AncCas-114) are effective to cleave dsDNA in human cellular systems for targeted gene knockout.

In one embodiment, AncCas proteins are substantially as effective as the control SpCas9 for lentiviral transfection cell editing.

In one embodiment, AncCas proteins is used in therapeutic development.

In one embodiment, AncCas-114 proteins is used in therapeutic development.

Turning now to FIG. 10, exemplary AncCas proteins can utilize a diversity of guide RNAs (e.g., sgRNAs).

In one embodiment, AncCas proteins can utilize a consensus guide RNA (e.g., sgRNA).

In one embodiment, the consensus guide RNA (e.g., sgRNA) is created using any suitable bioinformatic approaches and rationale design methods known in the art.

In one embodiment and as shown in FIG. 10, a consensus candidate sgRNA for AncCas-114 is disclosed (SEQ. ID. NO. 11). The consensus candidate sgRNA is created using any suitable bioinformatic approaches and rationale design methods.

In one embodiment, to create the consensus sgRNA, extant Cas9 sequences is accumulated, and guide RNAs (gRNAs) identified using any suitable methods known in the art. Next, related Cas9 species gRNAs is aligned (as shown in FIG. 10A) and a consensus sequence was identified. FIG. 10A shows the alignment of AncCas-114 related ExtCas sgRNA sequences identified by reference genomic sequence location. FIG. 10B depicts the resulting inferred sgAncCas114 that is used for testing.

In one embodiment, the consensus sgRNA for AncCas proteins includes a sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to at least one sequence of SEQ. ID. NO. 11 and functional fragments thereof.

In one embodiment, the consensus sgRNA for AncCas proteins includes a sequence wherein the sequence has a sequence similarity of at least about 95% with respect to at least one of a sequence of SEQ. ID. NO. 11 and functional fragments thereof.

In one embodiment, the method for identifying and creating novel ancestral AncCas proteins comprises identifying AncCas proteins having the consensus sgRNA for AncCas proteins including a sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to at least one of sequence of SEQ. ID. NO. 11 and functional fragments thereof.

In one embodiment, the method for identifying and creating novel ancestral AncCas proteins comprises identifying AncCas proteins having the consensus sgRNA for AncCas proteins including a sequence wherein the sequence is at least about 95% with respect to at least one of a sequence of SEQ. ID. NO. 11 and functional fragments thereof.

TABLE 2

| SEQ. ID. | | |
|---|---|---|
| NO. | Nucleotide sequence | |
| 11 | 5′- | RNA |
| | GGGCGAGGAGCUGUUCACCGGUUUUAGAGCUGGAAACAGCAAGUUAAAAUAAGGCU | |
| | UUGUCCGUACACAACUUGAAAAAGUGGCACCGAUUCGGUGCUU-3′ | |
| 12 | 5′- | RNA |
| | GGGCGAGGAGCUGUUCACCGCGUUUUAGAGCUAGAAAUAGCAAGUUAAAAUAAGGG | |
| | UAGUCCGUUAUCAACUUGAAAAAGUGGCACCGAGUCGGUGCUU-3′ | |
| 13 | 5′-GGGCGAGGAGCUGUUCACCGCGUUUUAGAGCUGGAAA | RNA |
| | CAGCAAGUUAAAAUAAGGCUUUGUCCGUACAC | |
| | AACUUGAAAAAGUGGCACCGAUUCGGUGCUUU-3′ | |
| 14 | 5′- | RNA |
| | NNNNNNNNNNNNNNNNNNNNGUUUUAGAGCUAGAAAUAGCAAGUUAAAAUAAGGC | |
| | UAGUCCGUUAUCAACUUGAAAAAGUGGCACCGAGUCGGUGC-3′ | |
| 15 | 5′- | RNA |
| | NNNNNNNNNNNNNNNNNNNNGUUUUAGAGCUAUGCUGUACAGCAUAGCAAGUUAAA | |
| | AUAAGGCUAGUCCGUUAUCAACUUGAAAAAGUGGCACCGAGUCGGUGC-3′ | |

Wherein N is any nucleotide.

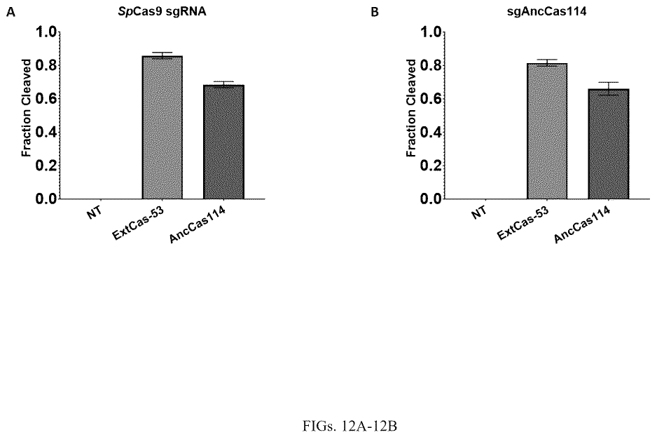

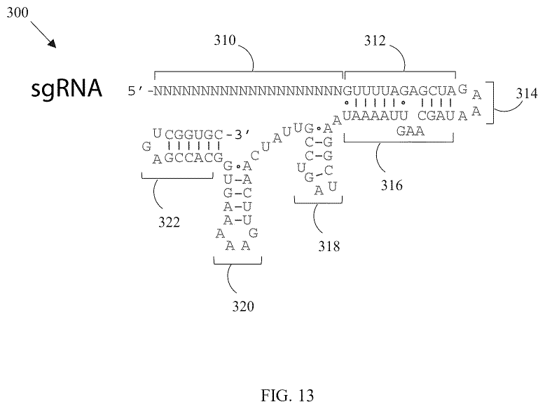

Turning to FIG. 11, sgRNA secondary structures for a SpCas9 sgRNA (FIG. 12A, SEQ. ID. NO. 12) and an inferred AncCas-114 sgRNA (FIG. 12B, SEQ. ID. NO. 13) are illustrated. In general, the secondary structure for a given sgRNA sequence is determined using methods known to those skilled in the art.

In one embodiment of the secondary structure of the inferred AncCas-114 sgRNA, differences is identified by the highlighted nucleotides in the inferred gRNA, thus exhibiting high conservation of both CRISPR RNA (crRNA) and trans-activating CRISPR RNA (tracrRNA) regions (FIG. 11).

In one embodiment, guide RNAs (gRNAs) for SpCas9 and sgAncCas114 is designed and transcribed according to any suitable methods known in the art.

FIGS. 11A-11B illustrate an exemplary secondary structure relationship between optimized Streptococcus pyogenes Cas9 and designed sgAncCas114. Black: crRNA target bound to target DNA. Blue: Truncated repeat. Grey: sgRNA tetraloop. Red: Anti-repeat RNA and tracrRNA. Green: Linker between stem loops 1 and 2. Yellow: Inferred AncCas114 sgRNA deviations from SpCas9 sgRNA.

In one embodiment, a composition is provided wherein the composition comprises at least one AncCas protein having a sequence wherein the sequence has a sequence similarity of at least about 25%, at least about 50%, at least about 70%, at least about 80%, at least about 90%, or at least about 95%, at least about 96%, at least about 97%, at least about 98%, or at least about 99% with respect to at least one sequence selected from the group consisting of SEQ. ID. NOs. 1-10 and functional fragments thereof and an sgRNA, the sgRNA having a sequence wherein the sequence has a sequence similarity of at least about 95%, at least about 96%, at least about 97%, at least about 98%, or at least about 99% with respect to at least one sequence selected from the group consisting of SEQ. ID. NOS. 11-22 and functional fragments thereof

In one embodiment, the resulting ancestral CAS (AncCas) proteins derived from the method for identifying and creating novel ancestral CAS (AncCas) proteins can successfully utilize SpCas9 guide RNAs.

In one embodiment, the resulting ancestral CAS (AncCas) proteins derived from the method for identifying and creating novel ancestral CAS (AncCas) proteins can exhibit a strong preference to NGG PAM regions similar to the control SpCas9 enzyme.

In one embodiment, the resulting AncCas is selected from the group consisting of AncCas-123, AncCas-121, AncCas-125, AncCas-120, AncCas-124, Anc-Cas-112, Anc-Cas-114, AncCas-87, Anc-Cas-84, and Anc-Cas-81. AncCas-123, AncCas-121, AncCas-125, AncCas-120, AncCas-124, Anc-Cas-112, Anc-Cas-114, AncCas-87, Anc-Cas-84, and Anc-Cas-81.

In one embodiment, the resulting AncCas is AncCas-114.

Turning now to FIG. 12, the method for identifying and creating novel ancestral CAS (AncCas) proteins comprises identifying AncCas proteins that can exhibit a similar cleavage efficiency to SpCas9. The AncCas proteins, for example AncCas-114, can exhibit a similar cleavage efficiency to SpCas9. As shown in FIG. 12, guide RNAs were tested using suitable in vitro cleavage assay methods with both the canonical SpCas9 sgRNA (FIG. 12A) and the inferred AncCas-114 sgRNA (FIG. 12B).

In one embodiment, SpCas9 and sgAncCas114 exhibit similar efficiency to cleave dsDNA using Cas9 enzymes.

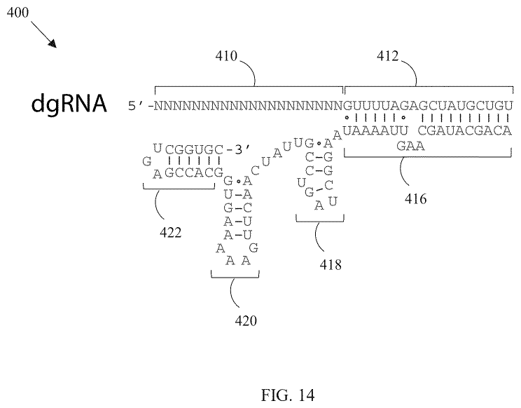

Referring now to FIGS. 13 and 14, two exemplary guide systems that is used by Cas proteins are provided. FIG. 13 shows a single fused guide sgRNA construct 300 (SEQ. ID. NO. 14).

In one embodiment the sgRNA construct 300 comprises a guide region (spacer) 310, a tracrRNA-pairing region (repeat) 312, a gRNA tetraloop 314, a crRNA-pairing region (anti-repeat) 316, a first stem loop 318, a second stem loop 320, and a third stem loop 322.

FIG. 14 shows a dual guide dgRNA construct 400 (SEQ. ID. NO. 15).

In one embodiment the dgRNA construct 400 comprises a guide region (spacer) 410, a tracrRNA-pairing region (repeat) 412, a crRNA-pairing region (anti-repeat) 416, a first stem loop 418, a second stem loop 420, and a third stem loop 422.

The exemplary AncCas proteins can demonstrate efficient cleavage using both the sgRNA (FIG. 13, SEQ. ID. NO. 14) and dgRNA (FIG. 14, SEQ. ID. NO. 15) in vitro. In one embodiment, the AncCas is AncCas-114.

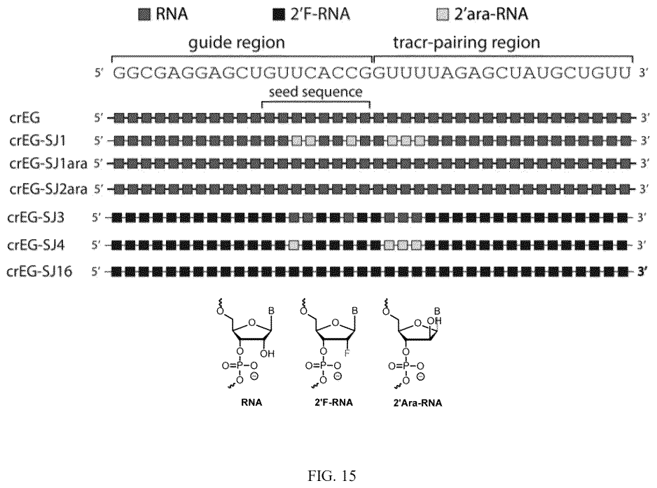

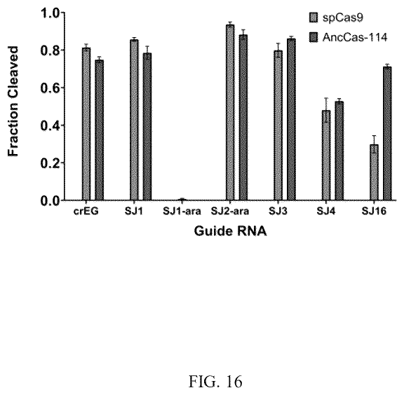

Referring now to FIG. 15, chemical modification schemes used for in vitro experiments with structures of 2′ F-RNA and 2′ Ara-RNA and the location of the modifications across the guide and tracr pairing region of the crRNA are provided. The exemplary AncCas proteins can function with any various chemically modified guide RNAs to cleave dsDNA. FIG. 16 discloses exemplary chemically modified guide RNAs including crEG (SEQ. ID. NO. 16), crEG-SJ1 (SEQ. ID. NO. 17), crEG-SJ1ara (SEQ. ID. NO. 18), crEG-SJ2ara (SEQ. ID. NO. 19), crEG-SJ3 (SEQ. ID. NO. 20), crEG-SJ4 (SEQ. ID. NO. 21), and crEG-SJ16 (SEQ. ID. NO. 22).

In one embodiment, the exemplary AncCas proteins can function with at least one of the chemically modified guide RNAs including crEG (SEQ. ID. NO. 16), crEG-SJ1 (SEQ. ID. NO. 17), crEG-SJ1ara (SEQ. ID. NO. 18), crEG-SJ2ara (SEQ. ID. NO. 19), crEG-SJ3 (SEQ. ID. NO. 20), crEG-SJ4 (SEQ. ID. NO. 21), and crEG-SJ16 (SEQ. ID. NO. 22).

In FIG. 15, red indicates RNA, black indicates 2′F-RNA, and yellow indicates 2′ara-RNA modifications.

Turning to FIG. 16, in vitro cleavage assay results for ExtCas-53 (SpCas9) and AncCas-114 using a catalogue of the chemically modified guides of FIG. 15 are provided.

In one embodiment, AncCas-114 can exhibit improved cleavage function with various chemically modified guide RNAs compared to the control SpCas9 enzyme.

In one embodiment, ancestral CAS proteins (AncCas proteins) is able to use more extensive guide modifications as shown by FIG. 16 where AncCas-114 can use in SJ16, a full 2′F-RNA molecule more efficiently than SpCas9 for fraction cleavage. Thus, amino acid sequence and structural deviation can allow for improved compatibility between enzyme-gRNA interactions especially in critical RNA base positions.

In one embodiment, any of the AncCas proteins described herein can exhibit a percent fraction cleavage of a target dsDNA comprising at least about 20%, at least about 40%, at least about 50%, at least about 60%, at least about 70%, at least about 80%, or at least about 90%.

In one embodiment, AncCas-114 can exhibit a percent fraction cleavage of a target dsDNA comprising at least about 20%, at least about 40%, at least about 50%, at least about 60%, at least about 70%, at least about 80%, or at least about 90%.

In one embodiment of the method for identifying and creating novel ancestral CAS (AncCas) proteins can identify and create at least one AncCas protein having a sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to at least one sequence selected from the group consisting of SEQ. ID. NOs. 1-10 and functional fragments thereof and an sgRNA, the sgRNA having a sequence wherein the sequence has a sequence similarity of at least about 95% with respect to at least one sequence selected from the group consisting of SEQ. ID. NOS. 11-22 and functional fragments thereof.

In one embodiment of the method for identifying and creating novel ancestral CAS (AncCas) proteins can identify and create at least one AncCas protein having a sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to at least one sequence selected from the group consisting of SEQ. ID. NOs. 1-10 and a functional fragment thereof and an sgRNA, the sgRNA having a sequence wherein the sequence has a sequence similarity of at least about 25%, at least about 50%, at least about 70%, at least about 80%, at least about 90%, at least about 95%, at least about 96%, at least about 97%, at least about 98%, or at least about 99% with respect to at least one sequence selected from the group consisting of SEQ. ID. NOS. 16-22 and functional fragments thereof.

In one embodiment, a composition is provided wherein the composition comprises at least one AncCas protein having a sequence wherein the sequence has a sequence similarity of at least about 25%, at least about 50%, at least about 70%, at least about 80%, at least about 90%, or at least about 95%, at least about 96%, at least about 97%, at least about 98%, or at least about 99% with respect to at least one sequence selected from the group consisting of SEQ. ID. NOs. 1-10 and functional fragments thereof and an sgRNA, the sgRNA having a sequence wherein the sequence has a sequence similarity of at least about 25%, at least about 50%, at least about 70%, at least about 80%, at least about 90%, at least about 95%, at least about 96%, at least about 97%, at least about 98%, or at least about 99%, with respect to at least one sequence selected from the group consisting of SEQ. ID. NOS. 11-22 and functional fragments thereof.

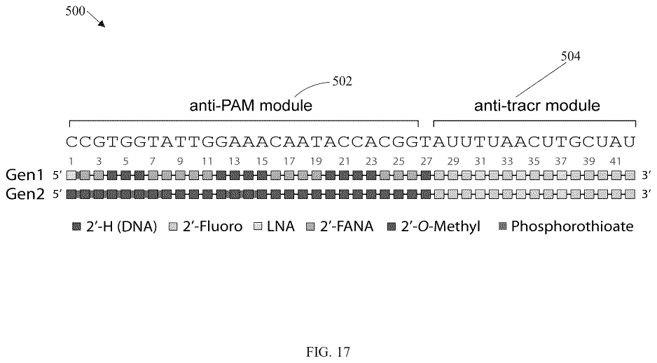

Referring to FIG. 17, an exemplary structure of a small nucleic acid-based inhibitor 500 (SEQ. ID. NO. 23) comprising an anti-PAM module 502 and an anti-tracr module 504 is provided.

In one embodiment, the AncCas proteins are provided in a composition with any SNuB system 500 as previously identified in the publication Barkau, C. L., O'Reilly, D., Rohilla, K. J., Damha, M. J. & Gagnon, K. T. Rationally Designed Anti-CRISPR Nucleic Acid Inhibitors of CRISPR-Cas9. Nucleic acid therapeutics 29, 136-147, the complete disclosures of which are hereby incorporated by reference in their entirety.

In one embodiment, Ancestral AncCas proteins can function with chemically modified guide RNAs and are turned off with chemically modified SNuBs.

In one embodiment, the AncCas-114 protein can function with chemically modified guide RNAs and is turned off with chemically modified SNuBs.

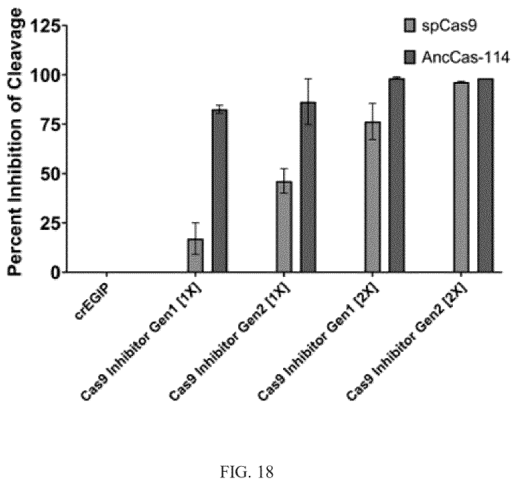

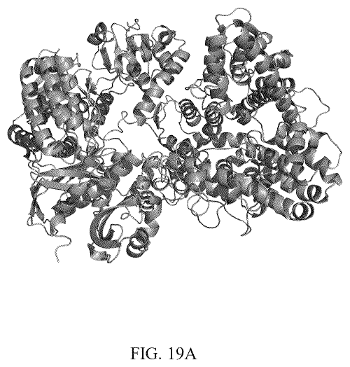

Turning to FIG. 18, in vitro cleavage assays show that AncCas proteins is used with various exemplary CRISPR SNuBs including two generations of SNuBs to inhibit cleavage activity. Furthermore, in vitro cleavage assays are used to evaluate SpCas9 and AncCas-114, wherein the cleavage assays is performed with inhibitor complexes and evaluated for inhibition potential.

In FIG. 18, activity of the inhibitor is reported as percent inhibition of cleavage normalized to crEGIP cleavage of the respective proteins. Cleavage and inhibition conditions are tested using 1:1 molar ratio of inhibitor to crEGIP guides (shown as [1×]) and 2:1 molar ratio of inhibitor to crEGIP guides (shown as [2×]).

FIG. 18 presents these results in an inverse manner of cleavage showing AncCas-114 sensitivity to lower doses of both inhibitors.

In one embodiment, the CRISPR-Cas inhibitors can exhibit varied degrees of sensitivity in ancestral Cas enzymes.

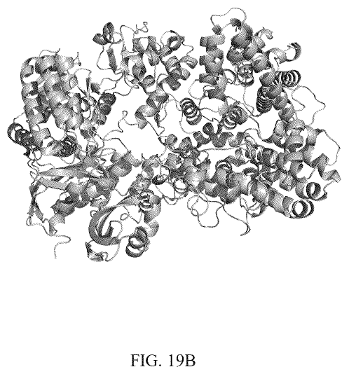

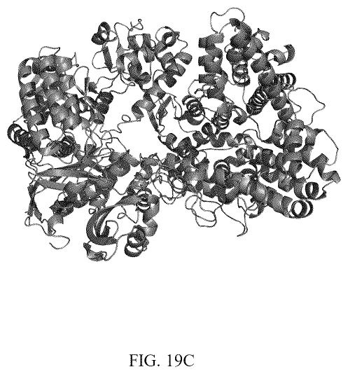

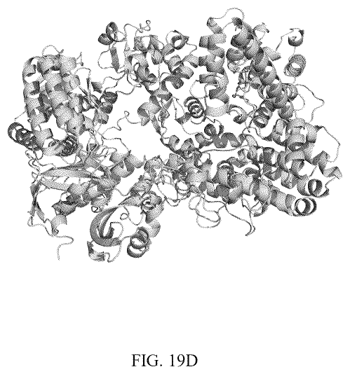

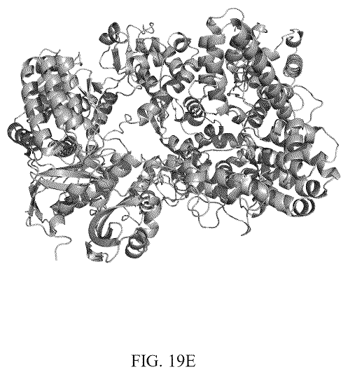

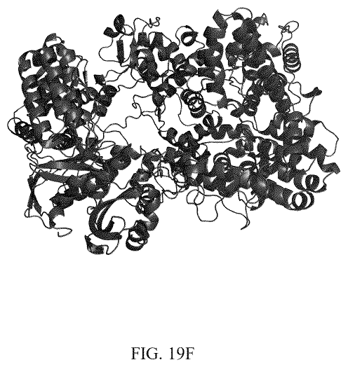

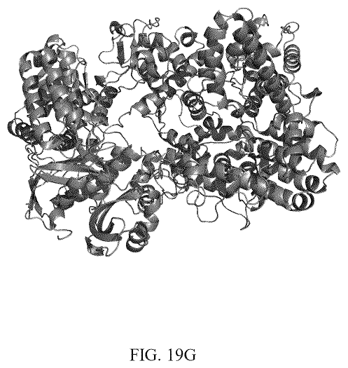

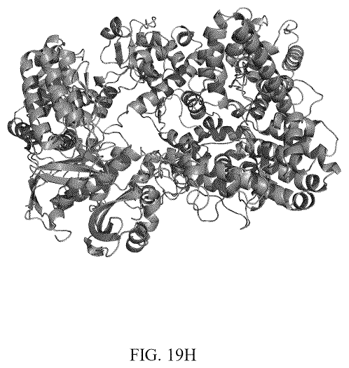

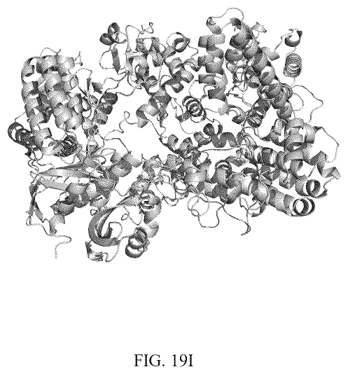

FIGS. 19-27 include illustrations of exemplary predicted protein folding structure of various exemplary AncCas proteins.

The following definitions and methods are provided to better define the present invention and to guide those of ordinary skill in the art in the practice of the present invention. Unless otherwise noted, terms are to be understood according to conventional usage by those of ordinary skill in the relevant art.

The term “construct” is understood to refer to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked.

An “expression vector”, “vector”, “vector construct”, “expression construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector is part of a plasmid, virus, or nucleic acid fragment. Typically, the expression vector includes a nucleic acid to be transcribed operably linked to a promoter.

Sequence similarity is understood as a measurement of how related biological sequences (e.g., DNA, RNA, or amino acid sequences) are when compared to each other. Sequence similarity can also be referred to as a percent sequence identity compared to a reference sequence.

Nucleotide and/or amino acid sequence identity percent (%), or sequence similarity %, is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) is calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

A fragment is understood as a sequence segment of a reference sequence. For example, a first sequence may have 100 nucleotides; a fragment of the sequence may include 90 nucleotides and 99% sequence similarity to the first segment.

A functional fragment is understood as a sequence fragment having a substantially similar function to a reference sequence. Similar function is understood to mean activity, for example cleavage activity. Substantially similar function of a functional fragment is at about 50%, at least about 60%, at least about 70%, at least about 80%, at least about 90%, at least about 95% activity compared to activity of the reference sequence.

In one embodiment, numbers expressing quantities of ingredients, properties such as molecular weight, reaction conditions, and so forth, used to describe and claim certain examples of the present disclosure are to be understood as being modified in some instances by the term “about.” In one embodiment, the term “about” is used to indicate that a value comprises the standard deviation of the mean for the device or method being employed to determine the value. In one embodiment, the numerical parameters set forth in the written description and attached claims are approximations that can vary depending upon the desired properties sought to be obtained by a particular example. In one embodiment, the numerical parameters should be construed in light of the number of reported significant digits and by applying ordinary rounding techniques. Notwithstanding that the numerical ranges and parameters setting forth the broad scope of some examples of the present disclosure are approximations, the numerical values set forth in the specific examples are reported as precisely as practicable. The numerical values presented in one embodiment of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In one embodiment, the terms “a” and “an” and “the” and similar references used in the context of describing a particular example (especially in the context of certain of the following claims) is construed to cover both the singular and the plural, unless specifically noted otherwise. When used in conjunction with the word “comprising” or other open language in the claims, the words “a” and “an” denote “one or more,” unless specifically noted. In one embodiment, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein are performed in any suitable order unless otherwise indicated herein or otherwise clearly contradicted by context. The use of any and all examples, or exemplary language (e.g., “such as”) provided with respect to certain examples herein is intended merely to better illuminate the present disclosure and does not pose a limitation.

Groupings of alternative elements or examples of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that all of the compositions and methods disclosed and claimed herein are made and executed without undue experimentation in light of the present disclosure. While the compositions and methods of this disclosure have been described in terms of preferred examples, it will be apparent to those of skill in the art that variations may be applied to the compositions and methods and in the steps or in the sequence of steps of the methods described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and this is considered to constitute examples of modes for its practice. However, those of skill in the art should, in light of the present disclosure, appreciate that many changes are made in the specific examples that are disclosed and still obtain a like or similar result without departing from the spirit and scope of the present disclosure.

### Example 1

### Ancestral Sequence Reconstruction and Selection Criteria.

A protein amino acid sequence BLAST of SpCas9 was used to gather 62 extant Streptococcus Cas9 protein sequences. An additional 4 Lactobacillus Cas9 protein sequences were included in the sequence list to be used as the outgroup phylogenetic root for evolutionary direction reference. These protein sequences were aligned using a multiple sequence alignment (MSA) and a phylogenetic analysis was performed with the Maximum Likelihood method and Whelan and Goldman (WAG)+Freq. model using MEGA X 37. The rates among sites were treated as a Gamma distribution with invariant sites using 0 Gamma categories (Gamma with invariant sites option). The MSA and phylogenetic tree analysis were utilized to infer ancestral Cas9 protein sequences using ProtASR2 38, producing 61 putative ancestral protein sequences.

Ancestral protein sequences were evaluated using in silico computational analysis programs including Alphafold2, SoluProt, Protein-Sol, and MEDUSA to measure success of prediction methods and variability of ancestral Cas9 protein structures and properties. Alphafold2 was used to predict 3D protein structures by inputting ancestral or extant protein sequences. Protein structures were aligned and evaluated using the molecular visualization system PyMOL. SoluProt was used as a sequence-based prediction tool for evaluating protein stability and solubility in Rosetta E. coli cells. Predicted protein structures were applied to Protein-Sol heatmap software to return heatmaps of protein stability and charge interactions across 91 combined pH and ionic strength conditions (FIG. 3). MEDUSA was used to evaluate protein flexibility by employing a convolutional network to assign flexibility classes for each residue across a protein sequence. These computational prediction models provided pre-screening methods which further guided the selection of ancestral Cas molecule selection for testing. The results from the computational analysis were compiled with bootstrap value outputs from the phylogenetic analysis to select twenty protein sequences with varied distance to SpCas9 and intriguing ancestral protein states. To evaluate the scope of the Cas9 ASR, 10 extant proteins (ExtCas) and 10 ancestral CAS proteins (AncCas) were selected for biochemical characterization.

### Example 2

Cloning into pET Vectors and Purification of Cas Proteins.

Ancestral and extant protein sequences were cloned into pET28a (+), kanamycin resistant protein expression vectors, by TWIST Biosciences with the coding regions flanked by 6×His, HA, and 3×NLS. To purify the Cas9 proteins, 100 ng of the expression plasmid DNA was transformed into 20 μL BL21 DE3 cells (NEB #C2527) and re-activated in 1 mL of LB media (Lennox formulation) at 30° C. A starter culture was prepared by adding LB media to the activated cells to a volume of 4.5 mL LB media containing 50 ug/mL kanamycin antibiotic. The starter culture was grown at 30° C. rotating at 200 rpm for 16 hours. To grow the main culture, the starter culture was added to a baffled flask containing 125 mL LB and 50 ug/mL kanamycin. The culture flask was incubated at 30° C. in a shaker incubator rotating at 200 rpm until the OD600 absorbance was approximately 0.6 at 600 nm. After reaching the stated growth value, IPTG was added to a final concentration of 0.4 mM and the culture was grown at 18° C. rotating at 150 μm for 16 hours. Cells were harvested by centrifugation and lysed using an Avestin Emulsiflex C3 high pressure homogenizer. Histidine tagged Cas9 proteins were purified using His-Tag isolation and pulldown magnetic beads (Thermo Fisher Scientific #10103D) following the manufacturers protocol. Protein elutions were concentrated and stored in a storage buffer consisting of 50 mM Sodium-Phosphate (pH 8.0), 300 mM NaCl, 0.01% Tween-20 (v/v), 1 mM EDTA, 2 mM DTT, and 50% glycerol. Protein concentrations were determined by a Nanodrop spectrophotometer (Thermo Fisher Scientific) using absorbances at 260 nm, calculated extinction coefficients, and the Beer-Lambert law. SDS-PAGE was used to determine success of purification and size pattern of ExtCas and AncCas proteins.

### Example 3

sgRNA Transcription and Purification.

sgRNA was prepared by T7 in vitro transcription with DNA templates synthesized by IDT. Single-stranded DNA templates were annealed to a T7 promoter oligo to generate double-stranded promoter regions, which support in vitro transcription by T7 RNA polymerase. Transcription reactions were performed by standard protocols for 2 hours. Briefly, reactions contained purified T7 RNA polymerase, 30 mM Tris (at pH 7.9), 12.5 mM NaCl, 40 mM MgCl2, 2% PEG8000, 0.05% Triton X-100, 2 mM spermidine, and 2.5 μM T7-DNA template. Afterward, the DNA template was degraded by the addition of 1 unit of DNase I for every 20 μL of reaction and incubated at 37° C. for 15 min. Reactions were gel-purified from denaturing polyacrylamide gels. Purified RNA was quantified by measuring absorbance at 260 nm and calculated extinction coefficients using nearest neighbor approximations and Beer's Law.

### Example 4

### In Vitro Cleavage Assay.

Preliminary dsDNA cleavage efficiency of AncCas and ExtCas enzymes was evaluated by in vitro cleavage assays. A 1 kb fragment of a target GFP gene was PCR-amplified from plasmid DNA (Addgene, 26777) and purified by magnetic bead separation. Cleavage assays were performed using both single guide RNA (sgRNA) and dual guide RNA (dgRNA) methods. dgRNA cleavage assays were performed in 40 μL reactions volumes by combining 160 ng target EGFP DNA, 10 μmol tracrRNA, 10 μmol crRNA, 1× cleavage buffer (20 mM Tris-HCl, pH 7.5, 100 mM KCl, 5% glycerol, 1 mM DTT, 0.5 mM EDTA, 2 mM MgCl2), 4 ug supplemental purified yeast tRNA, and 10 μmol of Cas9 enzyme. sgRNA cleavage assays were performed following the dgRNA protocol, replacing 10 μmol tracrRNAs and 10 μmol of crRNA with 10 μmol sgRNA. The reaction was incubated at 37° C. for 2 hours and stopped by adding 2% LiClO4 in acetone. The reaction was precipitated overnight at −20° C. and centrifuged. Precipitations were washed once with acetone, centrifuged, air dried, and resuspended in 13 μL ddH2O. Resuspended reactions were treated with 10 ug of RNase A and incubated at 37° C. for 20 minutes. Reactions were then treated with 20 ug of Proteinase K and incubated for 20 minutes at 37° C. Gel loading dye was added to the reactions and resolved on a 1.5% agarose gel stained with ethidium bromide. The gel was imaged on a BioRad ChemiDoc MP Imaging System and cleavage fractions were analyzed using ImageJ software (v1.54i).

### Example 5

### PAM Finding Assay.

Protospacer adjacent motif (PAM) sequence preference was determined by in vitro cleavage and next-gen sequencing methods. Duplex DNA with a 5-nucleotide region of randomized, complementary base pairs was ordered as a DNA oligonucleotide and amplified into duplex DNA by PCR with Q5 DNA polymerase (NEB #M0491). The primer nearest the predicted cleavage site terminus was blocked at the 5′ end with a carbon spacer chemistry to prevent adapter ligation during sequencing library preparation. The other primer, distal from the cleavage site, included a 5′ phosphate. Thus, in this example, only cleavage products that unblock the dsDNA substrate were carried forward during library preparation. Purified AncCas proteins were used in an in vitro cleavage assay following the previously described method, applying the randomized PAM sequence DNA target. The cleaved DNA library was prepared for sequencing by Oxford Nanopore Technologies (ONT) ligation-based sequencing methods (SQK-NBD114.24) following the manufacturers protocol. Sequencing results were analyzed, and sequence logos were constructed to identify PAM preferences which fell within the randomized target region 43.

### Example 6

### Chemically Modified RNA and Inhibitor Synthesis.

Inhibitor molecules and crRNAs were commercially synthesized by Integrated DNA Technologies (IDT) or custom synthesized. Custom synthesis used standard phosphoramidite solid-phase conditions. Syntheses were performed on an Applied Biosystems 3400 or Expedite DNA Synthesizer at a 1 micromole scale using Unylink CPG support (ChemGenes). All phosphoramidites were prepared as 0.13 M solutions in acetonitrile (ACN), except DNA, which was prepared as 0.1 M solutions. 5-Ethylthiotetrazole (0.25 M in ACN) was used to activate phosphoramidites for coupling. Detritylations were accomplished with 3% trichloroacetic acid in CH2Cl2 for 110 s. Capping of failure sequences was achieved with acetic anhydride in tetrahydrofuran (THF) and 16% N-methylimidazole in THF. Oxidation was done using 0.1M 12 in 1:2:10 pyridine: water: THF. Coupling times were 30 minutes for 2′F-RNA, and 50 minutes for LNA phosphoramidites. Deprotection and cleavage from the solid support was accomplished with 3:1:0.2 NH4OH:EtOH:DMSO at 65° C. for 16 h. Crude oligonucleotides were purified by anion exchange HPLC on an Agilent 1200 Series Instrument using a Protein-Pak DEAE 5 PW column (7.5×75 mm) at a flow rate of 1 mL/min. The gradient was 0-24% solution 1M LiClO4 over 30 min at 60° C. Samples were desalted on NAP-25 desalting columns according to manufacturer protocol. Modified crRNAs were prepared for RNP assembly by heating to 95° C. then placing on ice to prevent formation of stable secondary structures.

In vitro cleavage reactions were performed using chemically modified guides to determine efficiency and applicability to AncCas enzymes. Methods followed previously described dgRNA cleavage assay protocol and a catalogue of 6 different modified guide RNAs (crEG-SJ1, crEG-SJ1ara, crEG-SJ2ara, crEG-SJ3, crEG-SJ4, crEG-SJ16). The modification schemes and location of modifications in the guide RNA sequence is found in FIG. 15.

In vitro cleavage reactions were also performed with Cas9 small nucleic acid-based inhibitors (SNuB) to determine compatibility with previously published inhibitor molecules in the publication Barkau, C. L., O'Reilly, D., Rohilla, K. J., Damha, M. J. & Gagnon, K. T. Rationally Designed Anti-CRISPR Nucleic Acid Inhibitors of CRISPR-Cas9. Nucleic acid therapeutics 29, 136-147, the complete disclosures of which are hereby incorporated by reference in their entirety.

Cleavage reactions were performed following the sgRNA cleavage protocol containing two generations of Cas9 inhibitors at a 1× and 2× concentration of guide to inhibitor as described in Barkau, C. L., O'Reilly, D., Rohilla, K. J., Damha, M. J. & Gagnon, K. T. Rationally Designed Anti-CRISPR Nucleic Acid Inhibitors of CRISPR-Cas9. Nucleic acid therapeutics 29, 136-147, the complete disclosures of which are hereby incorporated by reference in their entirety.

### Example 7

Sub-Cloning into Lentiviral Vectors.

AncCas and ExtCas proteins which exhibited efficient cleavage of target DNA during in vitro cleavage assays and RNP assembled cell-based editing were subcloned into lentiviral vectors for transfection and expression in mammalian cell models. The coding sequence of the AncCas and ExtCas proteins were PCR amplified using Q5 Polymerase and purified by magnetic bead separation. pLJM1-EGFP (Addgene, 19319) was used as the lentiviral vector where the EGFP sequence was removed by restriction digestion and replaced by Infusion cloning methods with the amplified Cas protein sequence. Cloning success was determined by next-gen sequencing across the Cas gene and cloned plasmids were used for cell-based editing experiments.

### Example 8

### Cell-Based Editing.

To determine the efficiency of Cas9 cleavage in cellular conditions, therefore identifying the use as a therapeutic, two methods of gene knockout were designed. Ribonucleoprotein (RNP) complexes were assembled by combining purified AncCas proteins with a guide RNA targeting a model EGFP gene expressed constitutively in model HE293T cells. The RNP was electroporated into the model cells using a Neon Transfection instrument (Thermo Fisher Scientific #NEON1S). Editing was quantified by the loss of EGFP fluorescence using flow cytometry.

Cell-based EGFP knockout was also performed using the subcloned AncCas into pLJM1 lentiviral vectors. Plasmids were electroporated into the EGFP expressing HEK293T cells and incubated to express the Cas9 protein. After 48 hours, a sgRNA was transfected using lipid nanoparticles (RNAiMAX) and incubated for 96 hours, replacing the media daily. After 96 hours, GFP knockout and was quantified using flow cytometry on a BD LSRFortessa X-20 Analyzer (BD Biosciences).

### Example 9

Inferring sgRNA of AncCas Enzymes.

Experiments demonstrated in this study used canonical SpCas9 dual guide and single guide RNA for mediating double stranded breaks by extant and ancestral Cas9s. To evaluate the prospect of guide RNA evolution and therefore a necessity to develop unique gRNAs for ancestral enzymes, bioinformatic investigation and design of an AncCas-114 sgRNA was performed. Repeat RNA and tracrRNA sequences were accumulated from a previously published manuscript, Dooley, S. K., Baken, E. K., Moss, W. N., Howe, A. & Young, J. K. Identification and Evolution of Cas9 tracrRNAs. The CRISPR journal 4, 438-447, the complete disclosure of which are hereby incorporated by reference in their entirety, in which publicly available databases and bacterial reference sequences were mined to produce Cas9 associated guide RNAs. Extant Cas9 species associated with AncCas-114 were compiled and the guide RNA sequences were accumulated from the dataset. Manual annotations were performed to produce sgRNAs with canonical sequences for ExtCas proteins. These sgRNA sequences were aligned and used to create a consensus sequence by Jalview (v2.11.3.3) 46. The consensus sgAncCas114 RNA and ExtCas-canonical guide RNA were prepared by T7 in vitro transcription with a ssDNA template synthesized by IDT as previously described. In vitro cleavage assays were performed following the previously described protocol to determine efficiency of target DNA cleavage using extant guide RNAs and the inferred sgAncCas114.

### Example 9

Antibody binding to AncCas proteins.

To assess whether AncCas proteins is recognized by antibodies, and thus provide a surrogate assay for immune evasion via sequence diversification, anti-Cas9 antibody binding assays were performed with Western Blot techniques using commercially available antibodies (Abcam, ab189380). 300 ng of purified protein samples were run on a 7.5% Tris-Glycine gel at 100V for 2 hours. Proteins were transferred to PVDF membrane according to standard Western Blot techniques. Anti-Cas9 antibody was used to probe the gel at a 1:2000 dilution and visualized under a BioRad ChemiDoc MP Imaging System. Antibody binding was quantified using ImageJ.

1. A composition comprising an AncCas protein, wherein the AncCas protein comprises an amino acid sequence having a sequence similarity of at least about 95% with respect to at least one sequence selected from the group consisting of SEQ. ID. NOs. 1-10 and functional fragments thereof.

2. The composition of claim 1, further comprising the AncCas protein wherein the protein has a predicted SP of about 0.2 to about 1.0.

3. The composition of claim 1, wherein the AncCas protein has a sequence similarity of at least about 70% with respect to SpCas9.

4. The composition of claim 3, further comprising a guide RNA (sgRNA) or dual-guide RNA (dgRNA).

5. The composition of claim 4, wherein the guide RNA comprises a chemically modified guide RNA.

6. The composition of claim 5, wherein the guide RNA comprises a nucleotide sequence having a sequence similarity of at least about 95% with respect to at least one sequence selected from the group consisting of SEQ. ID. NOS. 16-22 and functional fragments thereof.

7. The composition of claim 6, wherein the composition is compatible with a small nucleic acid-based inhibitor (SNuB).

8. The composition of claim 7, wherein the SNuB comprises a nucleotide sequence having a sequence similarity of at least about 95% with respect to at least one sequence selected from the group consisting of SEQ. ID. NO. 23 and functional fragments thereof.

9. The composition of claim 8, wherein the composition selectively alters activity of at least one AncCas protein-guide RNA system.

10. A method for identifying and creating novel ancestral Cas (AncCas) proteins comprising:
performing ancestral sequence reconstruction;
performing a computational analysis of predicted sequences and their folded structures;
conducting in vitro biochemical assays; and
conducting in vivo cell-based assays.

11. The method of claim 10, wherein said performing ancestral sequence reconstruction comprises:
accumulating and aligning extant Cas sequences;
generating a phylogenetic tree; and
using an ASR algorithm software.

12. The method of claim 11, wherein said generating a phylogenetic tree comprises a phylogenetic analysis including protein sequences comprising between 5 and 5,000,000 Cas protein sequences.

13. The method of claim 12, wherein said performing a computational analysis of predicted sequences comprises at least one of:
predicting protein structure;
predicting protein solubility when expressed in bacterial or mammalian host cells
predicting protein stability and charge; and
predicting the flexibility of residues in the resulting protein.

14. The method of claim 13, wherein said conducting an in vitro biochemical assay comprises at least one of:
cloning and purification of a candidate AncCas sequence into a protein expression vector;
conducting a PAM finding assay;
assessing chemically modified guide cleavage activity; and
assessing inhibition from studied small nucleic acid-based inhibitors (SNuBs).

15. The method of claim 14, wherein said conducting an in vivo cell-based assay comprises at least one of:
performing transfection of assembled RNP complexes;
performing cloning of the selected AncCas gene into a lentiviral vector; and performing transfection of the AncCas encoded plasmid for fluorescence reporter gene knockdown or endogenous gene knockdown.

16. The method of claim 13, wherein at least one of the resulting AncCas proteins comprises an amino acid sequence having a sequence similarity of at least about 95% with respect to at least one sequence selected from the group consisting of SEQ. ID. NOs. 1-10 and functional fragments thereof.

17. The method of claim 13, wherein the AncCas protein has a predicted SP of about 0.2 to about 1.0.

18. The method of claim 13, wherein the AncCas protein has a sequence similarity of at least about 70% with respect to SpCas9.

19. A method of selectively modulating gene activity comprising administering a composition comprising a AncCas protein and a guide RNA.

20. The method of claim 19, wherein the AncCas protein comprises an amino acid sequence having a sequence similarity of at least about 95% with respect to at least one sequence selected from the group consisting of SEQ. ID. NOs. 1-10 and functional fragments thereof.


SEQUENCE LISTING (27 sequences)

SEQ ID NO: 1 - protein, 1358 aa, unidentified
MKKPYSIGLDIGTNSVGWAVITDDYKVPAKKMKVLGNTDKQYIKKNLLGALLFDSGETAEARRLKRTARRRYTRRRNRIRYLQEIFAEEMNKVDESFFHRLDDSFLVPEDKRGERHPIFGNLAEEVAYHENFPTIYHLRKHLADSPEKADLRLVYLALAHIIKFRGHFLIEGDLNSENTDVQKLFNEFVEVYDKTFEESHLSEQTVDVSAILTEKISKSRRLENLLKHFPNEKKNGLFGNFLALSLGLQPNFKSNFDLSEDAKLQFSKDTYEEDLENLLGQIGDEYADLFVAAKNLYDAILLSGILTVNDNSTKAPLSASMVKRYEEHQEDLAQLKEFIRENLPDKYKEIFSDKSKNGYAGYIEGKTSQEDFYKYLKGILSKIDGSEYFLEKIDREDFLRKQRTFDNGSIPHQIHLQEMHAILRRQGEYYPFLKENQEKIEKILTFRIPYYVGPLARGNSRFAWLSRKSDEKITPWNFDEVVDKESSAEAFIERMTNYDLYLPDEKVLPKHSLLYEKFTVYNELTKVKYVTEQGKAQFFDANMKQEIFDGLFKENRKVTKKKLMDYLDKEFDEFRIVDLTGLDKENKAFNASLGTYHDLLKIIKDKDFLDNPENEDILEDIVHTLTLFEDREMIKQRLAKYSDLFDKKVLKKLARRHYTGWGRLSAKLINGIRDKQSRKTILDYLIDDGYSNRNFMQLINDDSLSFKEEIAKAQVIGDTDDLKQVVQDLAGSPAIKKGILQSIKIVDELVKVMGHNPENIVIEMARENQTTNRGRRNSQQRLKRLEDAIKNLGSNILKEHPVDNQQLQNDRLFLYYLQNGKDMYTGEPLDIDRLSQYDIDHIIPQAFIKDDSIDNRVLTSSAKNRGKSDNVPSLEVVRKMKAFWQQLLNAKLISQRKFDNLTKAERGGLTEDDKAGFIKRQLVETRQITKHVARILDERFNTELDENGKRIRKVKIITLKSNLVSQFRKDFELYKVREINDYHHAHDAYLNAVVGTALLKKYPKLAPEFVYGDYPKYNSYKERGKATEKMFFYSNIMNFFKTEVKLADGTVVERPMIEVNEETGEIVWDKEKDFATVRKVLSYPQVNIVKKTEVQTGGFSKESILPKGNSDKLIPRKTKYWDPKKYGGFDSPTVAYSVLVVADVEKGKAKKLKTVKELVGITIMERSSFEKNPVAFLEKKGYRNIQEDTIIKLPKYSLFELENGRRRLLASAGELQKGNEMVLPQHLVTLLYHAQRINKTNEPEHLEYVEQHRHEFDELLNYIIDFAEKYVLAEKNLEKIKELYSKNEDASIEELASSFINLLTFTALGAPAAFKFFGTNIPRKRYTSTTECLNATLIHQSITGLYETRIDLSKLGED

SEQ ID NO: 2 - protein, 1362 aa, unidentified
MKKPYSIGLDIGTNSVGWAVITDDYKVPAKKMKVLGNTDKHFIKKNLLGALLFDSGTTAEDRRLKRTARRRYTRRRNRLRYLQEIFSEEMSKVDSSFFHRLDDSFLVPEDKRGSRYPIFGTLAEEKEYHENFPTIYHLRKHLADSKEKADLRLIYLALAHMIKYRGHFLYEESFDSKNNDIQKIFNEFISIYDNTFEGSSLSGQNAQVEAILTDKISKSAKRERILKLFPDEKSTGLFSEFLKLIVGNQADFKKHFDLEEKAPLQFSKDTYDEDLENLLGQIGDDFADLFLVAKKLYDAILLSGILTVTDVSTKAPLSASMIERYENHQKDLAALKQFIKNNLPEKYNEVFSDQSKDGYAGYIDGKTTQEAFYKYIKNLLSKFEGADYFLEKIEREDFLRKQRTFDNGSIPHQIHLQEMNAILRRQGEHYPFLKENKEKIEKILTFRIPYYVGPLARGNRDFAWLTRNSDQAIRPWNFEEVVDKASSAEDFINKMTNYDLYLPEEKVLPKHSLLYETFAVYNELTKVKFIAEGLRDYQFLDSGQKKQIVNLLFKEKRKVTEKDIIHYLHNVDGYDGIELKGIDKQFNASLSTYHDLLKIIKDKDFMDDPENEAILENIVHTLTIFEDREMIKQRLAQYDSIFDKKVIKALTRRHYTGWGKLSAKLINGIRDKQTGKTILDYLIDDGEINRNFMQLINDDSLSFKETIQKAQVIGETDNIKQVVQELPGSPAIKKGILQSIKIVDELVKVMGHNPESIVVEMARENQTTNQGRRNSQQRLKRLEDSLKNLGSNILKEHPVDNSQLQNDRLFLYYLQNGKDMYTGEALDINQLSSYDIDHIIPQAFIKDDSLDNRVLVSSAKNRGKSDDVPSLEVVQKRKAFWQQLLDSKLISERKFNNLTKAERGGLTEDDKAGFIKRQLVETRQITKHVAQILDARFNTEVTEKDKKNRTVKIITLKSNLVSNFRKEFELYKVREINDYHHAHDAYLNAVVAKALLKKYPKLEPEFVYGDYPKYNSYRERKKATEKVFFYSNIMNFFKTEVKLADGTVVKREQIEVNEETGEIVWNKEKHFATVRKVLSYPQVNIVKKTEVQTVGQNGGLFDNNIVPKGKVVNADKLIPIKSGLWDPEKYGGYARPTIAYSVLVIADIEKGKAKKLKTVKEMVGITIMDKKKFEKNPIAYLEEKGYKNINKNNIIKLPKYSLFEFENGRRRLLASSKELQKGNELVLPYHLTTLLYHAKRINKISEPEHLEYVEKHRNEFEELLNTIIEFSKKYVLAKANVEKIKQLYFDNEDADIEQLANSFISLLTFTSLGAPAAFKFFGVDISRSRYTSVSSCLNATLIHQSITGLYETRIDLSKLGED

SEQ ID NO: 3 - protein, 1358 aa, unidentified
MKKPYSIGLDIGTNSVGWAVITDDYKVPAKKMKVLGNTDKEYIKKNLLGALLFDSGETAEDRRLKRTARRRYTRRRNRILYLQEIFSEEMSKVDDSFFHRLDDSFLVPEDKRGSRYPIFGNLAEEKAYHENFPTIYHLRKHLADSKEKADLRLVYLALAHMIKYRGHFLIEGDFDSKNNDIQKIFQEFLEVYDNTFENSHLSEQNAQVEAILTDKISKSAKKERILKLFPNEKSTGLFSEFLKLIVGNQADFKKHFDLEEKAPLQFSKDTYDEDLENLLGQIGDDYADLFLAAKKLYDAILLSGILTVTDVSTKAPLSASMIQRYEEHQEDLAQLKQFIRNNLPEKYNEVFSDQSKNGYAGYIDGKTNQEAFYKYLKNLLSKFEGADYFLEKIEREDFLRKQRTFDNGSIPHQIHLQEMRAILRRQGEYYPFLKENKEKIEKILTFRIPYYVGPLARGNSDFAWLTRKSDEKITPWNFEEVVDKESSAEAFINRMTNYDLYLPEEKVLPKHSLLYETFTVYNELTKVKFIAEGLRDYQFLDSNQKKEIVNLLFKEKRKVTEKDIIDYLHKVDGYDGIELKGIDKQFNASLSTYHDLLKIIKDKDFLDDPENEAILEDIVHTLTIFEDREMIKQRLAKYDDIFDKKVLKKLTRRHYTGWGKLSAKLINGIRDKQSGKTILDYLIDDGNSNRNFMQLINDDALSFKEEIQKAQVIGDTDNIKQVVQDLPGSPAIKKGILQSIKIVDELVKVMGHNPESIVVEMARENQTTNQGRRNSQQRLKRLEDSLKNLGSNILKEHPVDNNQLQNDRLFLYYLQNGKDMYTGEALDIDRLSSYDIDHIIPQAFIKDDSIDNRVLVSSAKNRGKSDDVPSLEVVKKRKAFWQQLLDSKLISQRKFDNLTKAERGGLTEDDKAGFIKRQLVETRQITKHVARILDERFNTEVDENNKKIRTVKIITLKSNLVSNFRKDFELYKVREINDYHHAHDAYLNAVVAKALLKKYPKLEPEFVYGDYPKYNSYRERKKATEKVFFYSNIMNFFKKEVKLADGTVVERPMIEVNEETGEIVWNKEKHFATVRKVLSYPQVNIVKKTEVQTGGFSKENILPKGKEVNSDKLIPRKTKYWDPKKYGGFDSPTVAYSVLVIADIEKGKAKKLKTVKELVGITIMEKSKFEKNPIAFLEEKGYRNIQEDNIIKLPKYSLFELENGRRRLLASAGELQKGNELVLPNHLVTLLYHAKRINKTSEPEHLEYVEKHRNEFEELLNVIIEFSEKYILAEANLEKIKELYQKNENADIEELASSFINLLTFTSLGAPAAFKFFGVNIPRKRYTSVSECLNATLIHQSITGLYETRIDLSKLGED

SEQ ID NO: 4 - protein, 1359 aa, unidentified
MKKPYSIGLDIGTNSVGWAVVTDDYKVPAKKMKVLGNTDKSHIKKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEDLEVLLAQIGDDYADLFLSAKKLYDSILLSGILTVTDVSTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKILTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKAFNASYGTYHDLRKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKPFWSKLLSAKLITQRKFDNLTKAERGGLTDDDKAGFIKRQLVETRQITKHVARILDERFNTELDENNKKIRQVKIVTLKSNLVSNFRKEFELYKVREINDYHHAHDAYLNAVVGKALLGVYPQLEPEFVYGDYPHFHGHKEENKATAKKFFYSNIMNFFKKDVVRTDGTIVERPMIEVNDENGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGITIMEKMTFERNPVAFLERKGYRNIQEENIIKLPKYSLFELENGRKRLLASARELQKGNEIVLPNHLGTLLYHAKNIHKVDEPKHLDYVDKHKDEFKELLDVVSNFSKKYTLAEGNLEKIKELYAQNNSEDIKELASSFINLLTFTAIGAPATFKFFDKNIDRKRYTSTTEILNATLIHQSITGLYETRIDLSKLGGD

SEQ ID NO: 5 - protein, 1363 aa, unidentified
MKKPYSIGLDIGTNSVGWAVITDDYKVPAKKMKVLGNTDKQYIKKNLLGALLFDSGETAEATRLKRTARRRYTRRRNRLRYLQEIFAEEMNKVDESFFHRLDESFLVPEDKRGERHPIFGNIAEEVAYHQKFPTIYHLRKHLADSSEKADLRLVYLALAHIIKFRGHFLIEGDLNAENTDVQKLFNDFVEVYDKTVEESHLSEITVDAAAILTEKISKSRRLENLIKHYPTEKKNTLFGNLIALSLGLQPNFKTNFQLSEDAKLQFSKDTYEEDLEELLGQIGDDYADLFVAAKNLYDAILLSGILTVNDNSTKAPLSASMVKRYEEHQEDLAQLKEFIKENAPDKYNEIFKDKSKNGYAGYIENKVKQEDFYKYLKGILSKIDGSEYFLDKIDREDFLRKQRTFDNGSIPHQIHLQEMHAILRRQGEYYPFLKENQDKIEKILTFRIPYYVGPLARKNSRFAWASYKSDEKITPWNFDEVVDKEKSAEKFITRMTSYDLYLPEEKVLPKHSLVYEKFTVYNELTKVKYVNEQGKAKFFDANMKQEIFDHVFKENRKVTKEKLLNYLNKEFEEFRIVDLTGLDKENKAFNASLGTYHDLKKILDKDFLDDKANEDIIEDIIHTLTLFEDREMIRQRLQKYSDLFTKKQLKKLERRHYTGWGRLSYKLINGIRNKETNKTILDYLIDDGYANRNFMQLINDDSLSFKEEIAKAQVIGDVDDLKQVVHDLAGSPAIKKGILQSVKIVDELVKVMGHNPENIVIEMARENQTTNRGRRNSQQRLKRLQDSLKNLGSKILNEKNVENQQLQNDRLFLYYLQNGKDMYTGEPLDIDHLSQYDIDHIIPQAFIKDDSIDNRVLTSSAKNRGKSDNVPSLEVVRKRKAYWQRLRKAKLISQRKFDNLTKAERGGLTEDDKAGFIKRQLVETRQITKHVAQILDARFNTERDENGKVIRDVKIITLKSNLVSQFRKDFELYKVREINDYHHAHDAYLNAVVGTALLKKYPKLAPEFVYGEYKKYNVRKLIAKERGKATAKKFFYSNLMNFFKTEVKYADGTVVERPVIETNEETGEIVWNKEKDFATVRKVLSYPQVNIVKKVEVQTGGFSKESILPKGDSDKLIPRKTKNVYWDPKKYGGFDSPTVAYSVLVVADVEKGKAKKLKTVKELVGISIMERSAFEKNPVAFLEKKGYRNIQEDTIIKLPKYSLFELENGRRRLLASAGELQKGNEMVLPQKLVTLLYHAHRINNSNEPEHLEYVEKHKEEFKELLNYIVEFAEKYVLAEKNLEKIQALYSKNDDASIEELASSFINLLTFTALGAPAAFKFLGAKIPRKRYTSTTECLNATLIHQSITGLYETRIDLSKLGED

SEQ ID NO: 6 - protein, 1362 aa, unidentified
MDKKYSIGLDIGTNSVGWAVITDDYKVPSKKFKVLGNTDRHSIKKNLIGALLFDSGETAEATRLKRTARRRYTRRKNRIRYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRHYTGWGRLSRKLINGIRDKQSGKTILDFLKSDGFANRNFMQLIHDDSLTFKEEIQKAQVSGQGDSLHEQIANLAGSPAIKKGILQTVKVVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKRFFYSNIMNFFKTEITLANGEIRKRPLIETNGETGEIVWDKGRDFATVRKVLSMPQVNIVKKTEVQTGGFSKESILSKGNSDKLIARKKDWDPKKYGGFGSPTVAYSVLVVAKVEKGKAKKLKSVKELIGITIMERSSFEKDPIDFLEAKGYKDVQKDLIIKLPKYSLFELENGRRRMLASAGELQKGNEMVLPSKLVTFLYHASHIEKSKGSQHQAYVEQHRHDLDEILEYISEFSKRYILADKNLSKVKSAFNKHEDYSISELASSIINLFTLTSLGAPAAFKFLDTTIDRKRYTSTKEVLDATLIHQSITGLYETRIDLSQLGGD

SEQ ID NO: 7 - protein, 1364 aa, unidentified
MEKKYSIGLDIGTNSVGWAVITDDYKVPSKKFKVLGNTDRQSIKKNLIGALLFDSGETAEATRLKRTARRRYTRRKNRIRYLQEIFANEMAKVDDSFFHRLEESFLVEEDKKHERHPIFGNLADEVAYHENYPTIYHLRKKLADSPEKADLRLIYLALAHIIKFRGHFLIEGDLNAENSDVDKLFYQLVQTYNQLFEENPLDASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNSEITKAPLSASMVKRYDEHHQDLALLKALVRQQLPEKYKEIFSDQSKNGYAGYIDGKASQEEFYKFIKPILEKMDGAEELLAKLNREDLLRKQRTFDNGSIPHQIHLGELHAILRRQEEFYPFLKDNREKIEKILTFRIPYYVGPLARGNSRFAWLTRKSEETITPWNFEEVVDKGASAQSFIERMTNFDENLPNEKVLPKHSLLYEYFTVYNELTKVKYVTEGMRKPEFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRHYTGWGRLSRKLINGIRDKQSGKTILDFLKSDGFANRNFMQLIHDDSLTFKEEIQKAQVSGQGDSLHEQIANLAGSPAIKKGILQTVKIVDELVKVMGHKPENIVIEMARENQTTQKGLKNSRERMKRIEEGIKELGSQILKEHPVENTQLQNEKLYLYYLQNGRDMYVDQELDINRLSDYDVDHIVPQSFIKDDSIDNKVLTSSDKNRGKSDNVPSEEVVKKMKSYWRQLLNAKLISQRKFDNLTKAERGGLSEADKAGFIKRQLVETRQITKHVAQILDSRMNTKYDENDKPIREVKVITLKSKLVSDFRKDFQFYKVREINNYHHAHDAYLNAVVGTALIKKYPKLESEFVYGDYKVYDVRKMIAKSEQEIGKATAKRFFYSNIMNFFKTEVKLANGEIRKRPLIETNGETGEIVWDKEKDFATVRKVLSMPQVNIVKKTEVQTGGFSKESILSKGNSDKLIPRKKNWDPKKYGGFGSPTVAYSVLVVAKVEKGKAKKLKSVKELVGITIMERSSFEKDPIAFLEAKGYKDIQKDLIIKLPKYSLFELENGRRRMLASAGELQKGNEMVLPQHLVTFLYHASHIEKTTGSENLAYIEQHRHEFDEILEYIIEFSERYILADKNLSKVKSAFNKHEDFSISELASSFINLFTFTSLGAPAAFKFLDTTIDQGRKRYTSTTEVLDATLIHQSITGLYETRIDLSQLGGD

SEQ ID NO: 8 - protein, 1363 aa, unidentified
MKKPYSIGLDIGTNSVGWAVITDDYKVPAKKMKVLGNTDRQSIKKNLIGALLFDSGETAEATRLKRTARRRYTRRKNRIRYLQEIFAEEMAKVDDSFFQRLEDSFLVPEDKKYERHPIFGNLADEVAYHENYPTIYHLRKKLADSPEKADLRLIYLALAHIIKFRGHFLIEGDLNSENTDVQKLFHQLVDTYNLLFEEDPLDTETVDAKAILTAKISKSRRLENLIAQIPGQKKNGLFGNLIALSLGLTPNFKSNFDLSEDAKLQLSKDTYEEDLDNLLAQIGDQYADLFLAAKNLSDAILLSDILTVNDESTKAPLSASMVKRYEEHQQDLALLKKLVREQLPEKYKEIFSDKSKNGYAGYIDGKTSQEEFYKYIKPILSKLDGAEEFLAKIDREDFLRKQRTFDNGSIPHQIHLQELHAILRRQEEYYPFLKDNQEKIEKILTFRIPYYVGPLARGNSRFAWLTRKSDEAITPWNFEEVVDKEASAQAFIERMTNFDLYLPNEKVLPKHSLLYEMFTVYNELTKVKYVTEGMTKPEFLSAEQKQAIVDLLFKTNRKVTVKQLKENYFKKIECFDSVDITGVEDRFNASLGTYHDLLKIIKDKDFLDNPENEDILEDIVLTLTLFEDREMIEKRLAKYADLFDKKVLKKLKRRHYTGWGRLSRKLINGIRDKQSGKTILDFLKADGFANRNFMQLINDDSLSFKEEIAKAQVIGQSDSLKEQVANLAGSPAIKKGILQTIKIVDELVKVMGHNPENIVIEMARENQTTAQGIKNSRQRMKRLEEVIKKLGSQILKEHPVENTQLQNDRLYLYYLQNGKDMYTGEELDIDNLSQYDIDHIIPQSFIKDDSIDNKVLTSSEKNRGKSDNVPSIEVVKKMKSYWQQLLNAKLISQRKFDNLTKAERGGLSESDKAGFIKRQLVETRQITKHVAQILDSRFNTELDENDKRIRKVKIITLKSKLVSDFRKDFGLYKVREINDYHHAHDAYLNAVVGTALLKKYPKLAPEFVYGDYKKYDLYKMIAKSDSERGKATAKMFFYSNIMNFFKTEVKLADGTIIKRPLIEVNEETGEIVWDKEKDFATVRKVLSYPQVNIVKKTEVQTGGFSKESILSKGNSDKLIPRKNNYWDPKKYGGFDSPTVAYSVLVVAKVEKGKAKKLKTVKELVGITIMERSSFEKNPIAFLEAKGYRDIQEHLIIKLPKYSLFELENGRRRLLASAGELQKGNEMVLPQHLVTFLYHASRIDKLTGSEHLEYVEQHRHEFDEILEYIIEFAERYILADKNLEKIKSTYNKKEDFSINELASSFINLFTFTALGAPAAFKFFDTTIDRKRYTSTTECLNATLIHQSITGLYETRIDLSQLGGD

SEQ ID NO: 9 - protein, 1359 aa, unidentified
MKKPYSIGLDIGTNSVGWAVITDDYKVPAKKMKVLGNTDKQYIKKNLIGALLFDSGETAEARRLKRTARRRYTRRRNRIRYLQEIFAEEMNKVDESFFHRLDDSFLVPEDKRYERHPIFGNLAEEVAYHENFPTIYHLRKHLADSPEKADLRLVYLALAHIIKFRGHFLIEGDLNSENTDVQKLFNEFVEVYDKLFEESHLSEQTVDVKAILTEKISKSRRLENLLAHFPNEKKNGLFGNFLALSLGLQPNFKSNFDLSEDAKLQFSKDTYEEDLENLLGQIGDEYADLFLAAKNLYDAILLSGILTVNDESTKAPLSASMVKRYEEHQEDLAQLKEFIRENLPDKYKEIFSDKSKNGYAGYIEGKTSQEDFYKYLKGILSKIDGSEYFLEKIDREDFLRKQRTFDNGSIPHQIHLQELHAILRRQGEYYPFLKENQEKIEKILTFRIPYYVGPLARGNSRFAWLSRKSDEKITPWNFDEVVDKEASAEAFIERMTNYDLYLPDEKVLPKHSLLYEKFTVYNELTKVKYVTEGQGKPQFFSANMKQEIFDGLFKENRKVTKKKLMDYLDKEFDEFRIVDITGLDKENKAFNASLGTYHDLLKIIKDKDFLDNPENEDILEDIVHTLTLFEDREMIKQRLAKYSDLFDKKVLKKLARRHYTGWGRLSRKLINGIRDKQSRKTILDYLIDDGYSNRNFMQLINDDSLSFKEEIAKAQVIGDTDDLKQVVADLAGSPAIKKGILQSIKIVDELVKVMGHNPENIVIEMARENQTTNRGRRNSQQRLKRLEDAIKNLGSNILKEHPVDNQQLQNDRLYLYYLQNGKDMYTGEPLDIDRLSQYDIDHIIPQSFIKDDSIDNRVLTSSAKNRGKSDNVPSLEVVRKMKAFWQQLLNAKLISQRKFDNLTKAERGGLTEDDKAGFIKRQLVETRQITKHVARILDERFNTELDENGKRIRKVKIITLKSNLVSQFRKDFGLYKVREINDYHHAHDAYLNAVVGTALLKKYPKLAPEFVYGDYPKYNSYKERGKATEKMFFYSNIMNFFKTEVKLADGTVVERPMIEVNEETGEIVWDKEKDFATVRKVLSYPQVNIVKKTEVQTGGFSKESILPKGNSDKLIPRKTKYWDPKKYGGFDSPTVAYSVLVVADVEKGKAKKLKTVKELVGITIMERSSFEKNPVAFLEKKGYRNIQEDTIIKLPKYSLFELENGRRRLLASAGELQKGNEMVLPQHLVTLLYHAQRINKTNEPEHLEYVEQHRHEFDELLDYIIDFAEKYVLAEKNLEKIKELYNKNEDASIEELASSFINLLTFTALGAPAAFKFFGTTIPRKRYTSTTECLNATLIHQSITGLYETRIDLSKLGED

SEQ ID NO: 10 - protein, 1361 aa, unidentified
MKKPYSIGLDIGTNSVGWAVITDDYKVPAKKMKVLGNTDKQYIKKNLIGALLFDSGETAEARRLKRTARRRYTRRRNRIRYLQEIFAEEMNKVDESFFHRLDDSFLVPEDKRYDRHPIFGTLAEEVAYHENFPTIYHLRKHLADSPEKADLRLVYLALAHIIKFRGHFLIEGDLNSENTDVQKLFQEFVEVYDKLFEESHLSEQTVDVKAILTEKISKSRRVENILALFPGLNEKKKGLFGQFLKLSLGLQANFKSIFDLSEDAKLQFSKDSYEEDLENLLGQIGDEYADLFLAAKNLYDAILLSGILTVNDESTKAPLSASMVKRYEEHQEDLAQLKEFIRENLPDKYKEIFSDKSKNGYAGYIEGKTSQEDFYKYLKGILSKVEGSEYFLEKIEREDFLRKQRTFDNGSIPHQIHLQELHAILRRQGKYYPFLKENKEKIEKILTFRIPYYVGPLARGNSRFAWLSRKSDEKIRPWNFDEVVDKEASAEAFIERMTNHDLYLPDEKVLPKHSLLYEKFTVYNELTKVRYVTEGQGKPQFFSANMKQEIFDHLFKEHRKVTKKKLMDYLDKEFDEFRIVDITGLDKENKAFNASLSTYHDLKKIIKDKDFLDNPENQDILEEIVHTLTVFEDREMIKQRLAKYSDLFDKKVLKKLARRHYTGWGRLSRKLINGIRDKQSRKTILDYLIDDGYSNRNFMQLINDDGLSFKEEIAKAQVIGDSDDLKQVVADLAGSPAIKKGILQSIKIVDELVKVMGYAPENIVIEMARENQTTNRGRRNSQQRLKRLEDAIKNLGSNILKEHPVDNQQLQNDRLYLYYLQNGKDMYTGEPLDIDRLSQYDIDHIIPQSFIKDDSIDNRVLTSSAKNRGKLDNVPSLEVVKKMKAFWQQLYNAKLISQRKFDNLTKAERGGLTEDDKAGFIKRQLVETRQITKHVARLLDERFNTELDENGKRIRTVKIITLKSNLVSQFRKDFGLYKVREINDYHHAHDAYLNAVVGKALLKKYPKLAPEFVYGEYPKYNSYKERGKATEKMFFYSNIMNFFKKEVKLADGTVVERPMIEVNEETGEIVWDKEKDFATVRKVLSYPQVNIVKKTEVQTGGFSKESILPKGNSDKLIPRKTKYWDPKKYGGFDSPTVAYSVLVVADVEKGKAKKLKTVKELVGITIMERSSFEKNPVAFLEKKGYRNIQEHTIIKLPKYSLFELENGRRRLLASAGELQKGNQMVLPQHLVTLLYHAQRINKTNEPEHLEYVEQHRHEFDELLDYIVEFAEKYVLAEKNLEKIKELYNKNEDASIEELASSFINLLTFTALGAPAAFKFFGTTIPRKRYTSTTECLNATLIHQSITGLYETRIDLSKLGED

SEQ ID NO: 11 - RNA, 101 nt, synthetic construct
  source 1..101: mol_type = other RNA
gggcgaggagctgttcaccgcgttttagagctggaaacagcaagttaaaataaggctttgtccgtacacaacttgaaaaagtggcaccgattcggtgcttt

SEQ ID NO: 12 - RNA, 99 nt, synthetic construct
  source 1..99: mol_type = other RNA
gggcgaggagctgttcaccgcgttttagagctagaaatagcaagttaaaataagggtagtccgttatcaacttgaaaaagtggcaccgagtcggtgctt

SEQ ID NO: 13 - RNA, 101 nt, synthetic construct; identical to SEQ ID NO: 11
  source 1..101: mol_type = other RNA

SEQ ID NO: 14 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 15 - RNA, 104 nt, synthetic construct
  source 1..104: mol_type = other RNA
nnnnnnnnnnnnnnnnnnnngttttagagctatgctgtacagcatagcaagttaaaataaggctagtccgttatcaacttgaaaaagtggcaccgagtcggtgc

SEQ ID NO: 16 - RNA, 38 nt, synthetic construct
  source 1..38: mol_type = other RNA
ggcgaggagctgttcaccggttttagagctatgctgtt

SEQ ID NO: 17 - RNA, 38 nt, synthetic construct; identical to SEQ ID NO: 16
  source 1..38: mol_type = other RNA
  modified_base 14: mod_base = OTHER; note = 2'-arabino ribonucleotide
  modified_base 15: mod_base = OTHER; note = 2'-arabino ribonucleotide
  modified_base 18: mod_base = OTHER; note = 2'-arabino ribonucleotide
  modified_base 21: mod_base = OTHER; note = 2'-arabino ribonucleotide
  modified_base 22: mod_base = OTHER; note = 2'-arabino ribonucleotide
  modified_base 23: mod_base = OTHER; note = 2'-arabino ribonucleotide

SEQ ID NO: 18 - RNA, 38 nt, synthetic construct; identical to SEQ ID NO: 16
  source 1..38: mol_type = other RNA

SEQ ID NO: 19 - RNA, 38 nt, synthetic construct; identical to SEQ ID NO: 16
  source 1..38: mol_type = other RNA

SEQ ID NO: 20 - RNA, 38 nt, synthetic construct; identical to SEQ ID NO: 16
  source 1..38: mol_type = other RNA
  modified_base 1: mod_base = OTHER; note = 2'-fluoro ribonucleotide
  modified_base 2: mod_base = OTHER; note = 2'-fluoro ribonucleotide
  modified_base 3: mod_base = OTHER; note = 2'-fluoro ribonucleotide
  modified_base 4: mod_base = OTHER; note = 2'-fluoro ribonucleotide
  modified_base 5: mod_base = OTHER; note = 2'-fluoro ribonucleotide
  modified_base 6: mod_base = OTHER; note = 2'-fluoro ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 12: mod_base = OTHER; note = 2'-fluoro ribonucleotide
  modified_base 11: mod_base = OTHER; note = 2'-fluoro ribonucleotide
  modified_base 13: mod_base = OTHER; note = 2'-fluoro ribonucleotide
  modified_base 16: mod_base = OTHER; note = 2'-fluoro ribonucleotide
  modified_base 17: mod_base = OTHER; note = 2'-fluoro ribonucleotide
  modified_base 19: mod_base = OTHER; note = 2'-fluoro ribonucleotide
  modified_base 20: mod_base = OTHER; note = 2'-fluoro ribonucleotide
  modified_base 24: mod_base = OTHER; note = 2'-fluoro ribonucleotide
  modified_base 25: mod_base = OTHER; note = 2'-fluoro ribonucleotide
  modified_base 26: mod_base = OTHER; note = 2'-fluoro ribonucleotide
  modified_base 27: mod_base = OTHER; note = 2'-fluoro ribonucleotide
  modified_base 29: mod_base = OTHER; note = 2'-fluoro ribonucleotide
  modified_base 28: mod_base = OTHER; note = 2'-fluoro ribonucleotide
  modified_base 30: mod_base = OTHER; note = 2'-fluoro ribonucleotide
  modified_base 31: mod_base = OTHER; note = 2'-fluoro ribonucleotide
  modified_base 32: mod_base = OTHER; note = 2'-fluoro ribonucleotide
  modified_base 33: mod_base = OTHER; note = 2'-fluoro ribonucleotide
  modified_base 34: mod_base = OTHER; note = 2'-fluoro ribonucleotide
  modified_base 35: mod_base = OTHER; note = 2'-fluoro ribonucleotide
  modified_base 36: mod_base = OTHER; note = 2'-fluoro ribonucleotide
  modified_base 37: mod_base = OTHER; note = 2'-fluoro ribonucleotide
  modified_base 38: mod_base = OTHER; note = 2'-fluoro ribonucleotide

SEQ ID NO: 21 - RNA, 38 nt, synthetic construct; identical to SEQ ID NO: 16
  source 1..38: mol_type = other RNA
  modified_base 1..13: mod_base = OTHER; note = 2'-fluoro ribonucleotide
  modified_base 15..20: mod_base = OTHER; note = 2'-fluoro ribonucleotide
  modified_base 24..38: mod_base = OTHER; note = 2'-fluoro ribonucleotide
  modified_base 14: mod_base = OTHER; note = 2'-arabino ribonucleotide
  modified_base 21..23: mod_base = OTHER; note = 2'-arabino ribonucleotide

SEQ ID NO: 22 - RNA, 38 nt, synthetic construct; identical to SEQ ID NO: 16
  source 1..38: mol_type = other RNA
  modified_base 1..38: mod_base = OTHER; note = 2'-fluoro ribonucleotide

SEQ ID NO: 23 - DNA, 42 bp, synthetic construct
  misc_feature 1..27: note = DNA
  misc_feature 28..42: note = RNA
  source 1..42: mol_type = other DNA
ccgtggtattggaaacaataccacggtattttaacttgctat

SEQ ID NO: 24 - protein, 1384 aa, unidentified
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVMDKHHHHHH

SEQ ID NO: 25 - protein, 1464 aa, unidentified
MGYPYDVPDYAYPYDVPDYAYPYDVPDYASGSTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNGNLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEGGSGSPAAKRVKLDGSGSPKKKRKVGSGSPAAKRVKLDLEHHHHHH

SEQ ID NO: 26 - protein, 1444 aa, unidentified
MGYPYDVPDYAYPYDVPDYAYPYDVPDYASGSDKKYSIGLDIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKVDLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGSPAAKRVKLDGSGSPKKKRKVGSGSPAAKRVKLDLEHHHHHH

SEQ ID NO: 27 - protein, 1718 aa, unidentified
MKSSHHHHHHHHHHGSSMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEENLYFQSNAMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN